(19) United States
(12) Patent Application Publication
(10) Pub. No.: US 2025/0058079 A1
(43) Pub. Date: Feb. 20, 2025

(54) EXPANDABLE MEDICAL ACCESS SHEATHS

(71) Applicant: EDWARDS LIFESCIENCES CORPORATION, Irvine, CA (US)

(72) Inventors: Anne Bernadette Aragon Alcasid, Costa Mesa, CA (US); Cristobal R. Hernandez, Santa Ana, CA (US)

(21) Appl. No.: 18/936,870

(22) Filed: Nov. 4, 2024

Related U.S. Application Data
(63) Continuation of application No. PCT/US2023/021784, filed on May 10, 2023.
(60) Provisional application No. 63/341,384, filed on May 12, 2022.

(51) Int. Cl. A61M 25/00

(52) U.S. Cl. CPC A61M 25/0054; A61M 2025/0024

(57) ABSTRACT

An expandable medical access sheath can comprise a hub comprising a hub delivery lumen extending therethrough. A shaft can extend distally from the hub and comprise a shaft delivery lumen extending therethrough and that is in fluid communication with the hub delivery lumen. The shaft can comprise an expandable shaft portion comprising a laced portion extending along a first partial circumferential portion of the expandable shaft portion, the laced portion comprising a cord configured to form lacing for the laced portion, and an elongate foldable portion extending along a second partial circumferential portion of the expandable shaft portion.

### CROSS REFERENCE TO RELATED APPLICATIONS

This application is a continuation of International Patent Application No. PCT/US2023/021784, filed May 10, 2023, which claims the benefit of U.S. Provisional Patent Application No. 63/341,384, filed May 12, 2022, the complete disclosures of which are hereby expressly incorporated by reference herein in their entireties for all purposes.

### BACKGROUND

The present disclosure generally relates to the field of minimally invasive transcatheter delivery of medical devices. Expandable medical access sheaths can be used in minimally invasive transcatheter medical procedures for delivery of medical devices. Minimally invasive transcatheter procedures can be used to treat a variety of conditions, including conditions of the heart.

### SUMMARY

Described herein are methods and devices related to expandable access sheaths used in minimally invasive medical procedures for transcatheter delivery of medical devices to a target location. The expandable access sheaths can include a shaft comprising an expandable shaft portion. The expandable shaft portion can comprise one or more laced portions and one or more elongate foldable portions each extending along non-overlapping circumferential portions of the expandable shaft portion. A laced portion can extend between two elongate reinforcing members. The laced portion can comprise a cord configured to be slidably disposed through a plurality of openings along a longitudinal dimension of each of the two elongate reinforcing members so as to lace together the two elongate reinforcing portions. Pulling on the cord can change a lateral dimension, such as a width, of the laced portion, thereby allowing adjustment in a distance between the two elongate reinforcing portions and a lateral cross-sectional size of the expandable shaft portion. Each of the elongate foldable portions can fold or unfold, and/or stretch, to decrease or increase a lateral cross-sectional size of the expandable shaft portion, respectively.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silico, or overlaid on one or more of the physical components. Virtual elements can be presented on any combination of screens, headsets, holographically, projected, loud speakers, headphones, pressure transducers, temperature transducers, or using any combination of suitable technologies.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 shows an expandable access sheath comprising at least a portion of a sheath shaft disposed within a femoral artery for minimally invasive transcatheter delivery of a medical device to a target heart location in accordance with one or more examples.

FIGS. 2-1A and 2-1B provide perspective and distal-end views, respectively, of the expandable access sheath described with reference to FIG. 1 in an expanded state in accordance with one or more examples.

FIGS. 2-2A and 2-2B provide perspective and distal end views, respectively, of the expandable access sheath described with reference to FIG. 1 in another expanded state in accordance with one or more examples.

FIGS. 2-3A and 2-3B provide perspective and distal end views, respectively, of the expandable access sheath described with reference to FIG. 1 in the first expanded state, with lacing of the laced portion in a tensioned configuration in accordance with one or more examples.

FIGS. 2-4A and 2-4B provide perspective and distal end views, respectively, of the expandable access sheath described with reference to FIG. 1 in a collapsed state in accordance with one or more examples.

FIGS. 2-5A and 2-5B provide perspective and distal end views, respectively, of the expandable access sheath described with reference to FIG. 1 in another collapsed state in accordance with one or more examples.

FIG. 3A provides a perspective view, and FIGS. 3B and 3C provide distal end views, of an example of an expandable access sheath comprising elongate foldable portions having elasticity in accordance with one or more examples.

FIGS. 4A and 4B provide perspective and distal end views, respectively, of an example of an expandable access sheath that includes a laced portion comprising lacing disposed over an elongate foldable portion in accordance with one or more examples.

FIGS. 5A and 5B provide perspective and distal end views, respectively, of an example of an expandable access sheath comprising two opposingly oriented laced portions in accordance with one or more examples.

FIGS. 6A and 6B provide perspective and distal end views, respectively, of an example of an expandable access sheath comprising three laced portions evenly distributed around a circumference of an expandable shaft portion of the expandable access sheath in accordance with one or more examples.

FIG. 7 provides a perspective view of an example of an expandable access sheath comprising an expandable shaft portion extending along a proximal portion of a shaft in accordance with one or more examples.

FIG. 8 provides a perspective view of an example of an expandable access sheath comprising a laced portion extending along a proximal portion of an expandable shaft portion of the expandable access sheath in accordance with one or more examples.

FIG. 9 provides a perspective view of an example of an expandable access sheath comprising a first laced portion extending around a first partial circumferential portion along a proximal portion of a shaft of the expandable access sheath and a second laced portion extending around a second partial circumferential portion along a distal portion of the shaft in accordance with one or more examples.

FIG. 10 is an example of a process flow diagram illustrating an example of a process for delivering a medical device using an expandable access sheath as described herein in accordance with one or more examples.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

Expandable access sheaths can be used in minimally invasive transcatheter procedures to facilitate delivery of medical devices. Described herein are methods and devices related to expandable access sheaths that include a shaft comprising an expandable shaft portion with one or more laced portions. The laced portion can comprise a cord configured to form lacing for the laced portion. The cord of the laced portion can be configured to lace together two elongate reinforcing portions. Portions of the cord can slidably pass through a plurality of openings along a longitudinal dimension of each of the elongate reinforcing portions such that a lateral dimension, such as a width, of the laced portion can be adjusted. For example, pulling on the cord can result in tensioning the cord and reducing a width of the laced portion, adjusting a distance between the two elongate reinforcing portions, and thereby adjusting a lateral cross-sectional size of the expandable shaft portion.

In some instances, the expandable shaft portion can comprise one or more elongate foldable portions. An elongate foldable portion can extend between two elongate reinforcing portions. A circumference of an expandable shaft portion can comprise one or more laced portions, one or more elongate foldable portions, and a plurality of elongate reinforcing portions. Each of the one or more laced portions and one or more elongate foldable portions can extend along a corresponding non-overlapping partial circumferential portion of the expandable shaft portion. For example, one or more laced portions, one or more elongate foldable portions and a plurality of elongate reinforcing portions can extend along respective partial circumferential portions of the expandable shaft portion to form a circumference of the expandable shaft portion. In some instances, both a laced portion and an elongate foldable portion can extend along the same circumferential portion. The elongate foldable portions can comprise flexible and/or foldable material such that the elongate foldable portions can assume various folded configurations or an unfolded configuration. The expandable shaft portion can assume configurations with smaller or larger lateral cross-sectional sizes due to folding and unfolding of the elongate foldable portions, respectively. The elongate reinforcing portions can comprise a material more rigid than that of the elongate foldable portions so as to provide column strength for the expandable shaft portion. The elongate reinforcing portions can provide mechanical strength along a longitudinal dimension of the expandable shaft portion. In some instances, one or more of the elongate foldable portions can comprise elasticity along a lateral dimension, for example allowing stretching along the lateral dimension. In some instances, the expandable shaft portion can comprise a corset configuration. The ability of the expandable shaft portion to collapse or assume various expanded states having various cross-sectional sizes can allow accommodation of medical devices of various sizes, while facilitating navigation of the expandable shaft portion through tortuous anatomical pathways and anatomical pathways having varying widths.

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

Although the expandable access sheaths described herein are described as being inserted into the femoral artery for delivery of medical devices to the heart, it will be understood that the expandable access sheaths can be inserted into any number of other vessels and/or lumens to facilitate delivery of medical devices to any number of other organs, chambers, vessels and/or lumens. In some instances, the expandable access sheaths can be inserted into another arterial access opening. In some instances, a venous access approach can be used. For example, the expandable access sheaths can be inserted into a femoral vein, internal jugular vein, and/or subclavian vein. The expandable access sheaths can be used in any number of different types of minimally invasive transcatheter procedures.

It will be understood that the expandable access sheaths can undergo various processes in preparation for their use in the procedures, including for example sterilization processes. For example, expandable access sheaths as described herein can be sterilized expandable access sheaths. Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silico, or overlaid on one or more of the physical components. Virtual elements can be presented on any combination of screens, headsets, holographically, projected, loud speakers, headphones, pressure transducers, temperature transducers, or using any combination of suitable technologies.

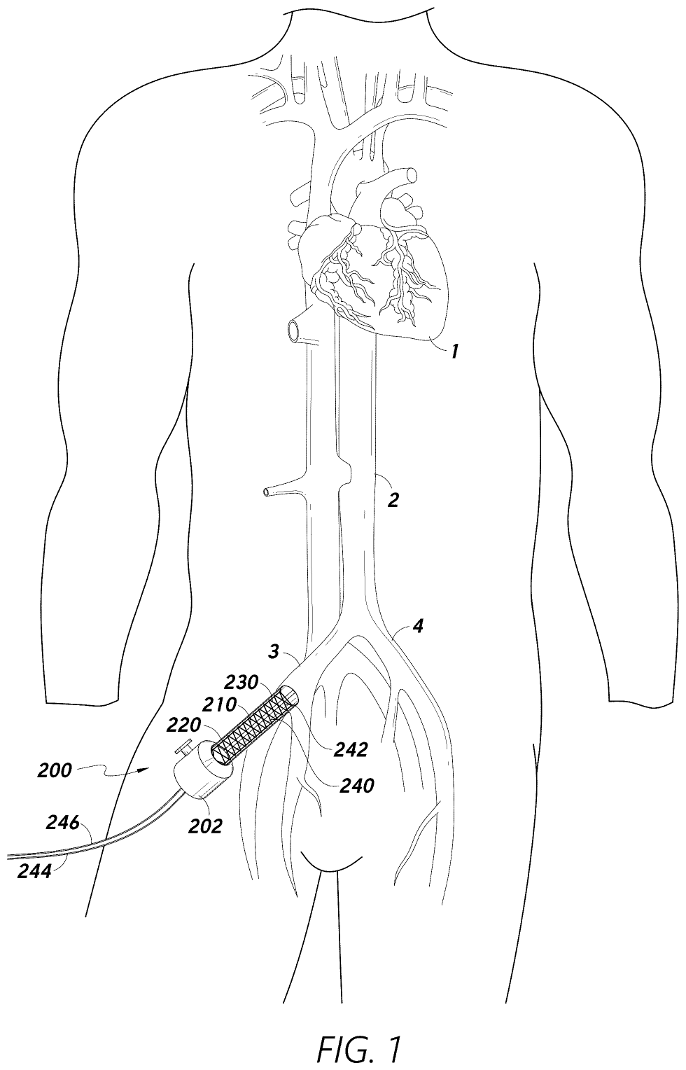

FIG. 1 shows an example of an expandable access sheath 200 disposed within a femoral artery 3 for minimally invasive transcatheter delivery of a medical device. The expandable access sheath 200 can be inserted into the right femoral artery 3 through an access opening formed thereon. It will be understood that the location of the access opening is shown for illustrative purposes only. The access opening can be made on any number of vessels and/or lumens of the patient, including for example, the left femoral artery 4. In some instances, another arterial access or a venous access can be used. A medical instrument, including a medical device, can be advanced through the expandable access sheath 200 after the expandable access sheath 200 is desirably positioned. The medical device or a delivery catheter carrying the medical device can be advanced through the expandable access sheath 200 to a target location. In some instances, the target location can be a target heart location. For example, the medical device can be advanced through the expandable access sheath 200 positioned within the right femoral artery 3 and into the aorta 2. In some instances, the medical device can be delivered into the heart 1 from the aorta 2. It will be understood that the delivery of the medical device to the heart 1 through the right femoral artery 3 and aorta 2 is for illustrative purposes only. The expandable access sheath 200 can be inserted into any number of other vessels and/or lumens for minimally invasive transcatheter delivery of a medical device to another target location via other delivery pathways.

As described in further detail herein, the expandable access sheath 200 can comprise a shaft 210 extending distally from a hub 202. The shaft 210 can comprise an expandable shaft portion 220 that includes a laced portion 230. The laced portion 230 can comprise a cord 240 configured to form the lacing for the laced portion 230. For example, the cord 240 can comprise a lacing portion 242 configured to form the laced portion 230. The cord 240 can comprise a first and second tail portion 244, 246 proximal of the lacing portion 242. An operator, such as a surgeon, can manipulate the first and second tail portions 244, 246, such as to tension the cord 240. In some instances, the first and second tail portions 244, 246 can comprise a portion configured to be disposed proximally of the hub 202 to allow manipulation of the cord 240 by the operator. A width of the laced portion 230 can be adjusted by pulling on the first and second tail portions 244, 246. Adjusting the width of the laced portion 230 can result in adjustment of a lateral cross-sectional size of the expandable shaft portion 220, such as a diameter of the expandable shaft portion 220. The lateral cross section can be perpendicular or substantially perpendicular to a longitudinal axis of the shaft 210. For example, the operator can pull on the first and second tail portions 244, 246 to tension the lacing portion 242 of the cord 240, such as to cinch the lacing, reducing the width of the laced portion 230. The width of the laced portion 230 can be a dimension perpendicular or substantially perpendicular to the longitudinal axis of the shaft 210. FIG. 1 shows the expandable shaft portion 220 in an expanded state. In some instances, the expandable access sheath 200 can be advanced into the femoral artery 3 while the laced portion 230 is in an expanded state. For example, a dilator can be preloaded and disposed through the shaft 210 while the shaft 210 is advanced to a desired position. After the shaft 210 is positioned at a desired position, the dilator can be withdrawn. The medical device or the delivery catheter carrying the medical device can subsequently be advanced through the shaft 210.

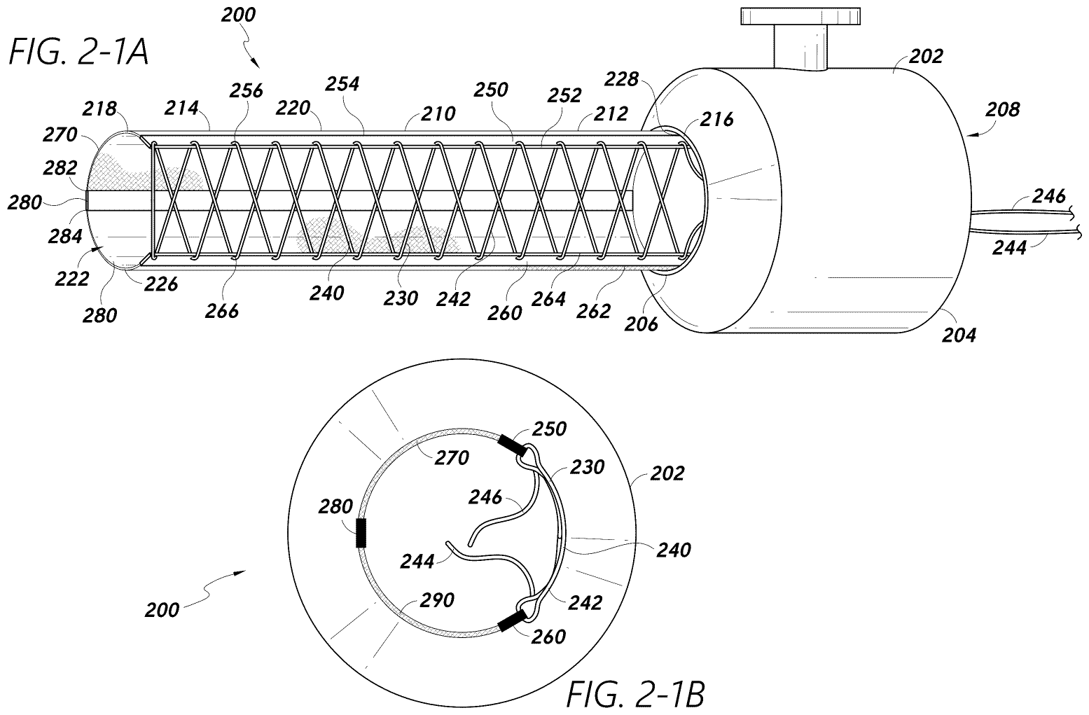

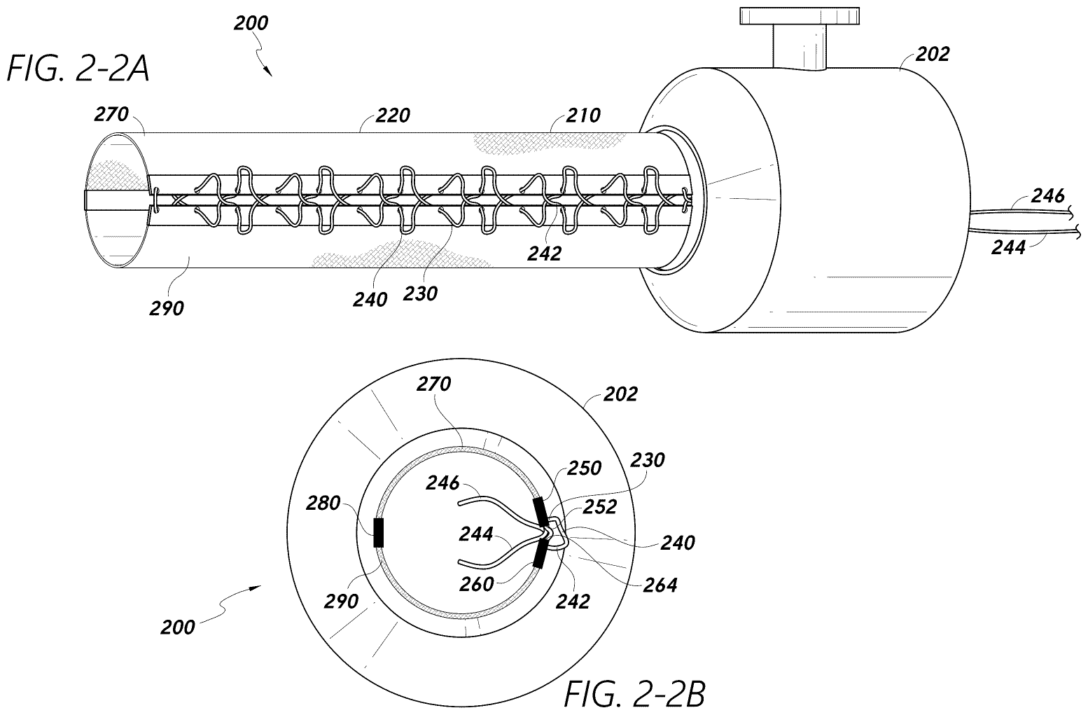

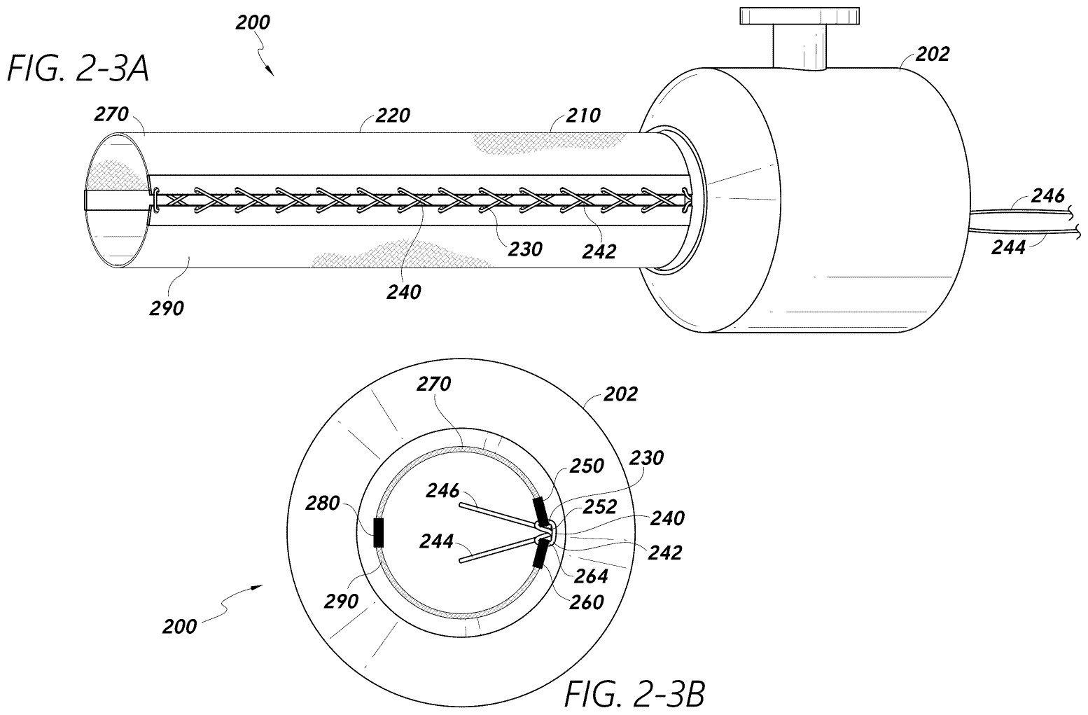

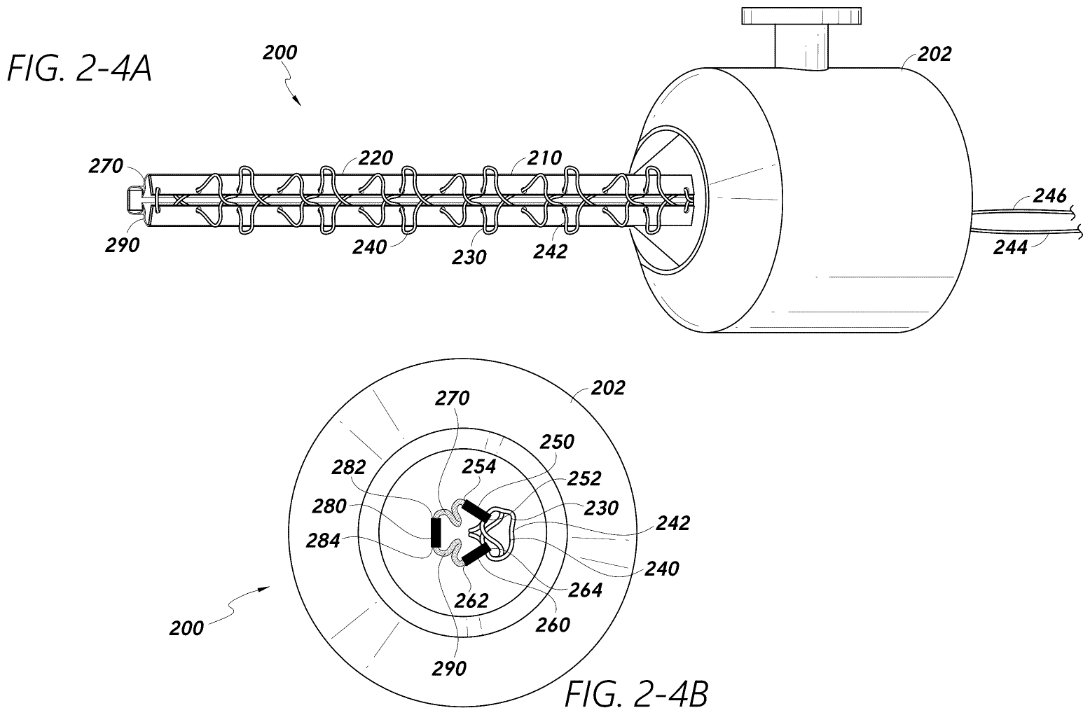

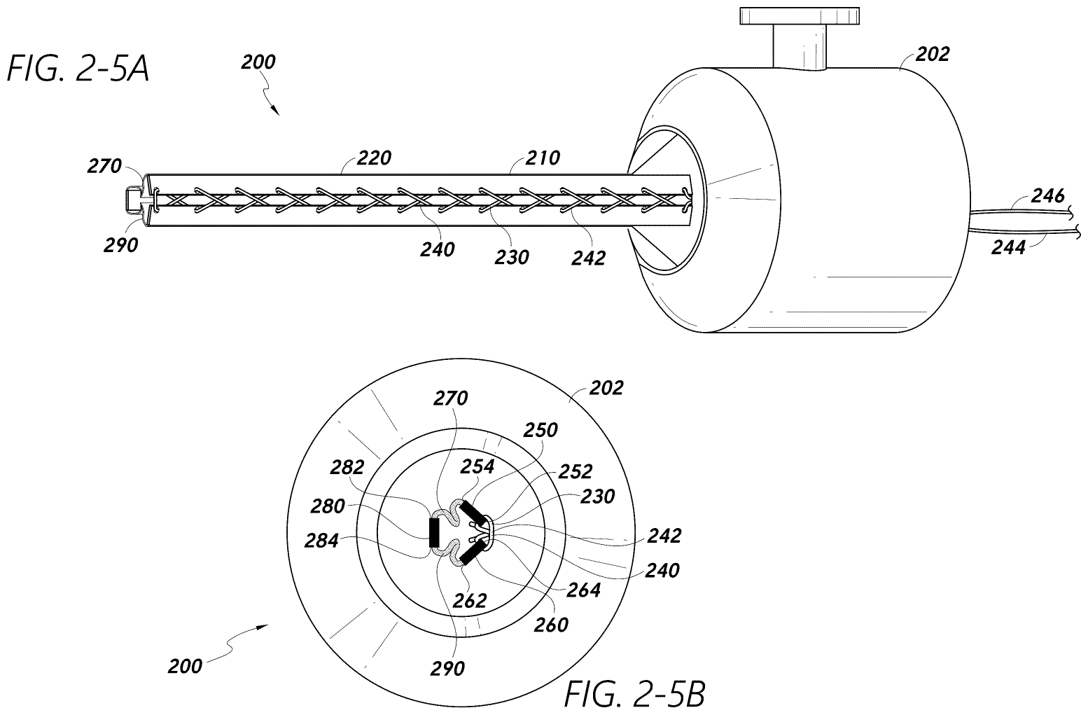

FIGS. 2-1A through 2-5B show various views of the expandable access sheath 200 described with reference to FIG. 1. FIG. 2-1A is a perspective view, and FIG. 2-1B is a distal end view, of a fully expanded expandable access sheath 200 described with reference to FIG. 1. The expandable shaft portion 220 is shown in an expanded state in FIGS. 2-1A and 2-1B. The expandable access sheath 200 can comprise a hub 202 and a shaft 210 extending distally from the hub 202. A proximal portion 212 of the shaft 210 can be associated with, such as being coupled to, a distal end 206 of the hub. The shaft 210 can extend distally from the hub 202, such as from the distal end 206 of the hub 202. A hub delivery lumen 208 can extend through the hub 202, from a proximal end 204 to the distal end 206 of the hub 202. The shaft 210 can comprise a shaft delivery lumen 222 extending therethrough. The shaft delivery lumen 222 can be in fluid communication with the hub delivery lumen 208. For example, a medical device can be advanced through the hub delivery lumen 208, then into and through the shaft delivery lumen 222 from the proximal portion 212 to the distal portion 214 of the shaft 210. At least a portion of the shaft 210 can comprise the expandable shaft portion 220. In some instances, the expandable shaft portion 220 can extend along an entire longitudinal dimension of the shaft 210. In some instances, the expandable shaft portion 220 can extend along an entire longitudinal dimension of the shaft 210 extending distally from the hub 202. The longitudinal dimension can extend along a direction parallel or substantially parallel to the longitudinal axis of the shaft 210. For example, the expandable shaft portion 220 can extend from a proximal end 216 to a distal end 218 of the shaft 210. A proximal end 228 of the expandable shaft portion 220 can be the proximal end 216 of the shaft 210. A distal end 226 of the expandable shaft portion 220 can be the distal end 218 of the shaft 210. The expandable shaft portion 220 can be configured to be radially expandable and/or collapsible. A lateral dimension of the expandable shaft portion 220 perpendicular or substantially perpendicular to the longitudinal axis of the shaft 210 can increase or decrease. For example, the expandable shaft portion 220 can be configured to expand radially such that lateral dimensions perpendicular or substantially perpendicular to, and that extend radially from, the longitudinal axis of the shaft 210 can increase.

A circumference of an expandable shaft portion can comprise one or more laced portions, one or more elongate foldable portions, and a plurality of elongate reinforcing portions, each of the one or more laced portions, one or more elongate foldable portions, and plurality of elongate reinforcing portions extending along a corresponding partial circumferential portion of the circumference. The expandable shaft portion can be configured to be in various states of expansion. For example, one or more of the laced portions can be in a collapsed or expanded configuration, and/or one or more of the elongate foldable portions can be in a folded or unfolded configuration, such that the expandable shaft portion can assume various configurations having different lateral cross-sectional sizes.

In some instances, the expandable shaft portion 220 can comprise a laced portion 230 extending along a first partial circumferential portion of the expandable shaft portion 220. The laced portion 230 can extend along an entire or substantially entire longitudinal dimension of the expandable shaft portion 220. The laced portion 230 can comprise a cord 240 configured to form lacing for the laced portion 230. A tension in the cord 240 of the laced portion 230 can be configured to be adjustable to change a lateral dimension, such as a width, of the laced portion 230. The cord 240 can comprise a lacing portion 242 configured to form the lacing, and a first and a second tail portion 244, 246 disposed proximally of the lacing portion 242. An operator, such as a surgeon, can manipulate the first and second tail portions 244, 246 to adjust the tension in the cord 240, such as to change the width of the laced portion 230. In some instances, the cord 240 can form lacing along an entire or substantially entire longitudinal dimension of the laced portion 230. The first and second tail portions 244, 246 can comprise respective portions configured to be disposed proximally of the hub 202. For example, the first and second tail portions 244, 246 can comprise a portion extending proximally from the proximal end 204 of the hub 202 to allow the operator to manipulate the first and second tail portions 244, 246.

The expandable shaft portion 220 can comprise a first and a second elongate reinforcing portion 250, 260 extending along a longitudinal dimension thereof. The laced portion 230 can extend between the first and second elongate reinforcing portions 250, 260. For example, the first and second elongate reinforcing portions 250, 260 can each comprise a first edge 252, 262 and a second edge 254, 264. The first edge 252 of the first elongate reinforcing portion 250 can be configured to be oriented toward the second edge 264 of the second elongate reinforcing portion 260. The second edge 254 of the first elongate reinforcing portion 250 can be configured to be oriented away from the second elongate reinforcing portion 260. The first edge 262 of the second elongate reinforcing portion 260 can be configured to be oriented away from the first elongate reinforcing portion 250. The cord 240 of the laced portion 230 can lace together the first edge 252 of the first elongate reinforcing portion 250 and the second edge 264 of the second elongate reinforcing portion 260. A tension in the cord 240 of the laced portion 230 can be configured to be adjustable to change a distance between the first and the second elongate reinforcing portions 250, 260, such as between the first edge 252 and the second edge 264.

In some instances, the first and the second elongate reinforcing portion 250, 260 can each comprise a plurality of openings 256, 266 along a longitudinal dimension thereof. The cord 240 can be configured to be passed through the plurality of openings 256, 266 to form the lacing for the laced portion 230. Respective portions of the lacing portion 242 of the cord 240 can be disposed through each of the openings 256, 266 to form the lacing of the laced portion 230. Respective portions of the cord 240 can be slidably disposed through the openings 256, 266 to facilitate adjustment in the distance between the first and second elongate reinforcing portions 250, 260. Adjusting the tension in the cord 240 can comprise sliding portions of the cord 240 through one or more of the openings 256, 266. For example, the operator can pull the first and second tail portions 244, 246 to cause portions of the cord 240 to slidably pass through the openings 256, 266 such that more of the cord 240 can be disposed proximally of the hub 202, reducing the distance between the first and second elongate reinforcing portions 250, 260. In some instances, as a medical device or a delivery catheter carrying the medical device is advanced through the shaft delivery lumen 222, a distance between the first and second elongate reinforcing portions 250, 260 can increase as portions of the cord 240 slidably pass through the openings 256, 266 such that less of the cord 240 is disposed proximally of the hub 202.

In the alternative, a plurality of loops can be coupled to each of the first edge 252 of the first elongate reinforcing portion 250 and the second edge 264 of the second elongate reinforcing portion 260. Respective portions of the cord 240 can be configured to be slidably passed through the plurality of loops to form the lacing.

In some instances, the expandable shaft portion 220 can comprise a first elongate foldable portion 270 extending along a second partial circumferential portion of the expandable shaft portion 220. The second partial circumferential portion can be another circumferential portion of the expandable shaft portion 220 that is non-overlapping with the first partial circumferential portion of the laced portion 230. The first elongate foldable portion 270 can extend between two elongate reinforcing portions. FIGS. 2-1A and 2-1B show the elongate foldable portion 270 extending between the second edge 254 of the first elongate reinforcing portion 250 and a first edge 282 of a third elongate reinforcing portion 280. The expandable shaft portion 220 can comprise a second elongate foldable portion 290 extending along a third partial circumferential portion of the expandable shaft portion 220. The third partial circumferential portion can be another circumferential portion of the expandable shaft portion 220 that is non-overlapping with the first and second partial circumferential portions. The laced portion 230, the first elongate foldable portion 270 and the second elongate foldable portion 290 can each form a respective portion of the circumference of the expandable shaft portion 220. The second elongate foldable portion 290 can extend between two elongate reinforcing portions, for example between the first edge 262 of the second elongate reinforcing portion 260 and a second edge 284 of the third elongate reinforcing portion 280. For example, the first elongate foldable portion 270 can extend from the second edge 254 of the first elongate reinforcing portion 250 to the first edge 282 of the third elongate reinforcing portion 280. The second elongate foldable portion 290 can extend from the first edge 262 of the second elongate reinforcing portion 260 to the second edge 284 of the third elongate reinforcing portion 280. The laced portion 230, first elongate reinforcing portion 250, first elongate foldable portion 270, third elongate reinforcing portion 280, second elongate foldable portion 290 and second elongate reinforcing portion 260 can be at respective positions around a circumference of the expandable shaft portion 220, including forming the circumference of the expandable shaft portion 220.

The expandable shaft portion 220 can assume different states of expansion. For example, the expandable shaft portion 220 can assume more or less expanded or collapsed states as the elongate foldable portions 270, 290 assume increasingly folded or unfolded configurations and as the laced portion 230 assumes wider or narrower widths. The ability of the expandable shaft portion 220 to assume various expanded or collapsed states having various cross-sectional sizes can facilitate accommodation of medical devices of various sizes. For example, FIGS. 2-1A and 2-1B show the expandable shaft portion 220 in a second expanded state. The first and second elongate foldable portions 270, 290 are each in an unfolded configuration and the laced portion 230 is in an expanded configuration. For example, in the second expanded state, the laced portion 230 can be configured to assume a fully expanded configuration, the width of the laced portion 230 being at a preselected maximum. The first and second elongate foldable portions 270, 290 can be in a fully unfolded configuration.

The expandable shaft portion 220 can assume states that are less expanded than that as shown in FIGS. 2-1A and 2-1B as the laced portion 230 assumes configurations with narrower widths, and as the first and second elongate foldable portions 270, 290 assume more folded configurations. FIG. 2-2A is a perspective view, and FIG. 2-2B is a distal end view, of the expandable access sheath 200 in a first expanded state, where the cord 240 of the laced portion 230 can comprise slack. In the first expanded state, the laced portion 230 can be in a collapsed configuration. The first and second elongate foldable portions 270, 290 can assume unfolded configurations. The first and second elongate foldable portions 270, 290 can be fully unfolded. In the example shown in FIGS. 2-2A and 2-2B, the cord 240 for the laced portion 230 can comprise slack. In some instances, the laced portion 230 can be provided in the collapsed configuration with slack in the cord 240 to facilitate expansion of the laced portion 230. As described herein, the laced portion 230 can assume intermediate configurations between the expanded configuration shown in FIGS. 2-1A and 2-1B and the collapsed configuration shown in FIGS. 2-2A, and 2-2B.

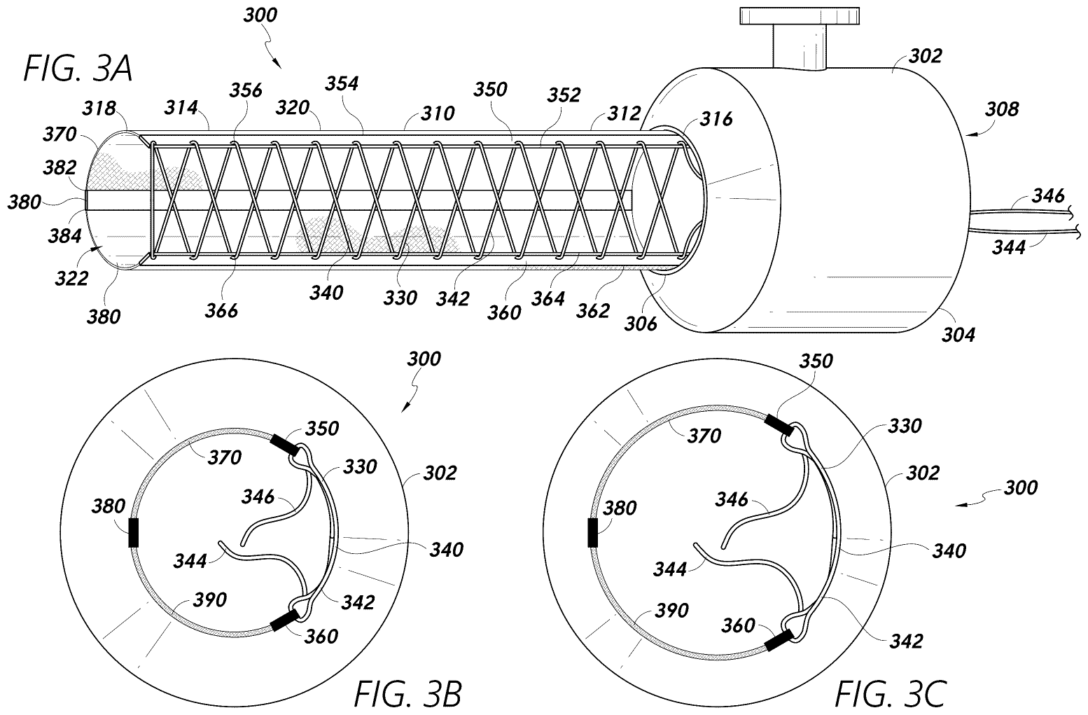

Referring to FIGS. 2-3A and 2-3B, the expandable access sheath 200 is shown in the first expanded state, while the lacing portion 242 of the laced portion 230 is in a tensioned configuration. In the tensioned configuration, the lacing portion 242 can be tensioned such that no or substantially no slack remains. For example, an operator can tug on the first and second tail portions 244, 246 to reduce slack in the lacing portion 242. While the laced portion 230 is in the collapsed configuration and the lacing portion 242 is in the tensioned configuration, a distance between the first edge 252 of the first elongate reinforcing portion 250 and the second edge 264 of the second elongate reinforcing portion 260 can be reduced or minimized. In some instances, the first edge 252 of the first elongate reinforcing portion 250 and the second edge 264 of the second elongate reinforcing portion 260 can be in contact with one another.

FIG. 2-4A is a perspective view, and FIG. 2-4B is a distal end view, of the expandable access sheath 200 in a collapsed state while the cord 240 of the laced portion 230 comprises slack. The first and second elongate foldable portions 270, 290 can assume folded configurations in the collapsed state. For example, the first and second elongate foldable portions 270, 290 can be fully folded. The slack in the laced portion 230 can facilitate expansion of the laced portion 230.

In some instances, a dilator can be preloaded and disposed through the shaft 210 while the shaft 210 is advanced to a desired position. The expandable shaft portion 220 can be in the second expanded state while the dilator is preloaded therethrough. After the shaft 210 is positioned at a desired position, the dilator can be withdrawn. After the dilator is withdrawn, the laced portion 230 can assume the collapsed configuration described with reference to FIGS. 2-4A and 2-4B. For example, the laced portion 230 can assume the collapsed configuration while comprising slack. The medical device or the delivery catheter carrying the medical device can subsequently be advanced through the shaft 210. The slack in the laced portion 230 can facilitate expansion of the laced portion 230 to accommodate the medical device or the delivery catheter carrying the medical device.

FIG. 2-5A is a perspective view, and FIG. 2-5B is a distal end view, of the expandable access sheath 200 in the collapsed state while the cord 240 of the laced portion 230 is in a tensioned configuration. As described, the first and second elongate foldable portions 270, 290 can assume folded configurations in the collapsed state, for example being fully folded. In some instances, the expandable access sheath 200 can assume the collapsed state shown in FIGS. 2-5A and 2-5B to facilitate withdrawal through an anatomical pathway. For example, the expandable access sheath 200 can be collapsed and the cord 240 can be pulled to remove any slack in the lacing portion 242, prior to withdrawing the expandable access sheath 200. While in the collapsed state, at least a portion of each of the elongate foldable portions 270, 290 can fold inward toward the longitudinal axis of the expandable shaft portion 220, and the laced portion 230 can assume a collapsed configuration. The edges 252, 254, 262, 264, 282, 284 of the elongate reinforcing portions 250, 260, 280 can be positioned closer to one another, such as compared to while the expandable shaft portion 220 is in a more expanded state.

The expandable access sheath 200 can assume intermediate expanded states in between the collapsed state and the first expanded state, and/or between the first expanded state and the second expanded state as described herein. Each of the elongate foldable portions 270, 290 can assume configurations having various degrees of folding. The laced portion 230 can assume intermediate states of expansion between the collapsed configuration and the expanded configuration described herein.

It will be understood that the number of laced portions, elongate foldable portions and elongate reinforcing portions described with reference to FIGS. 2-1A through 2-5B are for illustrative purposes only. Although FIGS. 2-1A through 2-5B show two elongate foldable portions, an expandable shaft portion can comprise more or fewer elongate foldable portions. For example, one elongate foldable portion extending between the second edge 254 of the first elongate reinforcing portion 250 and the first edge 262 of the second elongate reinforcing portion 260. The laced portion 230, the first and second elongate reinforcing portions 250, 260 and the one elongate foldable portion can form a circumference of the expandable shaft portion. Alternatively, three or more elongate foldable portions can be between the second edge 254 of the first elongate reinforcing portion 250 and the first edge 262 of the second elongate reinforcing portion 260, where each elongate foldable portion extends between a respective pair of elongate reinforcing portions. As described in further detail herein, an expandable shaft portion can comprise more than one laced portion.

FIG. 3A is a perspective view, and FIGS. 3B and 3C are distal end views, of an example of an expandable access sheath 300 comprising an expandable shaft portion 320 with at least one elongate foldable portion that can comprise elasticity along a lateral dimension. The expandable access sheath 300 can comprise a shaft 310 comprising the expandable shaft portion 320. The expandable shaft portion 320 can comprise a laced portion 330 extending along a first partial circumferential portion of the expandable shaft portion 320. A first elongate foldable portion 370 can extend along a second partial circumferential portion and a second elongate foldable portion 390 can extend along a third partial circumferential portion of the expandable shaft portion 320. The second and third partial circumferential portions do not overlap with one another and do not overlap with the first circumferential portion around which the laced portion 330 extends. In some instances, both the first and the second elongate foldable portions 370, 390 can comprise elasticity along a lateral dimension. The lateral dimension can extend along a direction perpendicular or substantially perpendicular to a longitudinal dimension of the elongate foldable portions 370, 390, such as to a longitudinal axis of the shaft 310. For example, the first and the second elongate foldable portions 370, 390 can reversibly stretch along a width. The elasticity of the first and the second elongate foldable portions 370, 390 can allow the expandable shaft portion 320 to assume a configuration with a wider diameter, such as compared to an expandable shaft portion comprising elongate foldable portions without elasticity. Both the first and second elongate foldable portions 370, 390 can stretch along a width to provide a larger lateral cross-sectional size, including a larger diameter, for the expandable shaft portion 320, such as compared to when the first and second elongate portions 370, 390 are not stretched. In alternative examples, only one of the first and the second elongate foldable portions 370, 390 comprises the elasticity along the lateral dimension. FIGS. 3A and 3B show the expandable shaft portion 320 in an expanded state where the first and the second elongate foldable portions 370, 390 are each in an unfolded and un-stretched configuration. FIG. 3C is a distal end view showing the first and the second elongate foldable portions 370, 390 each in an unfolded and stretched configuration.

The expandable access sheath 300 can have one or more other features of the expandable access sheath 200 described with reference to FIG. 2. For example, the shaft 310 can extend distally from a distal end 306 of a hub 302. The hub can comprise a hub delivery lumen 308 in fluid communication with the shaft delivery lumen 322. For example, a medical device can be advanced through the hub delivery lumen 308, then into and through the shaft delivery lumen 322 from the proximal portion 312 to the distal portion 314 of the shaft 310. A proximal portion 312 of the shaft 310 can be associated with the hub 302. The expandable shaft portion 320 can extend along an entire length of the shaft 310 disposed distally of the distal end 306 of the hub 302, such as from a proximal end 316 to a distal end 318 of the shaft 310. In some instances, the expandable shaft portion 320 can comprise a cord 340 configured to form lacing for the laced portion 330. The cord 340 can comprise a lacing portion 342 configured to form the lacing, and a first and a second tail portion 344, 346 disposed proximally of the lacing portion 342. The laced portion 330 can extend between a first and a second elongate reinforcing portion 350, 360, such as between and coupling a first edge 352 of the first elongate reinforcing portion 350 and a second edge 364 of the second elongate reinforcing portion 360. In some instances, the first and the second elongate reinforcing portion 350, 360 can each comprise a plurality of openings 356, 366 along a longitudinal dimension thereof through which respective portions of the lacing portion 342 of the cord 340 can be disposed through to form the lacing of the laced portion 330. The first and second tail portions 344, 346 can comprise a portion extending through the hub delivery lumen 308 and a portion disposed proximally of a proximal end 304 of the hub 302 to allow an operator to manipulate the first and second tail portion 344, 346 when adjusting a tension in the cord 340.

The first elongate foldable portion 370 and the second elongate foldable portion 390 can each extend between two elongate reinforcing portions. FIGS. 3A and 3B show the first elongate foldable portion 370 extending between the second edge 354 of the first elongate reinforcing portion 350 and a first edge 382 of a third elongate reinforcing portion 380. The second elongate foldable portion 390 can extend between the first edge 362 of the second elongate reinforcing portion 360 and a second edge 384 of the third elongate reinforcing portion 380. For example, the laced portion 330, first elongate foldable portion 370 and second elongate foldable portion 390 can define respective portions of a shaft delivery lumen 322 extending through the shaft 310.

The expandable shaft portion 320 can assume increasingly more expanded states as the laced portion 330, and the first and second elongate foldable portions 370, 390 assume expanded, unfolded and/or unfolded and stretched configurations. For example, while the expandable shaft portion 320 is in a first expanded state, the laced portion 330 can be in an expanded configuration and both the first and second elongate foldable portions 370, 390 can be in a folded configuration. While the expandable shaft portion 320 is in a second expanded state, the laced portion 330 can be in the expanded configuration and one of the first or second elongate foldable portions 370, 390 can be in an unfolded configuration. The laced portion 330 can be in the expanded configuration and both the first and second elongate foldable portions 370, 390 can be in an unfolded configuration while the expandable shaft portion 320 is in a third expanded state. While in a fully expanded state, such as a fourth expanded state, the laced portion 330 can be in the expanded configuration and both the first and second elongate foldable portions 370, 390 can in an unfolded and stretched configuration. In some instances, while the expandable shaft portion 320 is in a collapsed state, the laced portion 330 can be in a collapsed configuration and both the first and second elongate foldable portions 370, 390 can be in a folded configuration. The lacing of the laced portion 330 can have slack or be in a tensioned state while the laced portion 330 is in the collapsed configuration. The expandable shaft portion 320 can assume various intermediate expanded states between the expanded and/or collapsed states described herein.

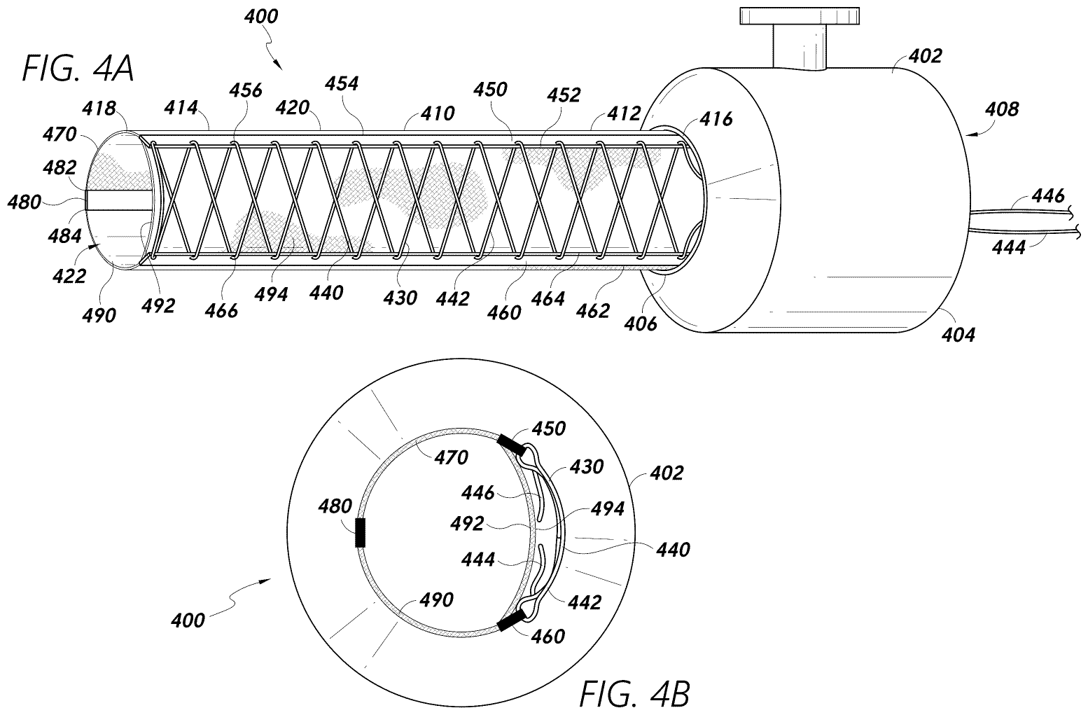

FIG. 4A is a perspective view, and FIG. 4B is a distal end view, of an example of an expandable access sheath 400 comprising an expandable shaft portion 420 with a laced portion 430 that includes both a first elongate foldable portion 492 and a cord 440 that forms lacing for the laced portion 430. The expandable access sheath 400 can comprise a shaft 410 comprising the expandable shaft portion 420. The expandable shaft portion 420 can comprise the laced portion 430 extending along a first partial circumferential portion of the expandable shaft portion 420. The cord 440 can be disposed over an externally oriented surface portion 494 of the elongate foldable portion 492. The laced portion 430 can extend between a first and a second elongate reinforcing portion 450, 460, such as between a first edge 452 of the first elongate reinforcing portion 450 and a second edge 464 of the second elongate reinforcing portion 460. A lacing portion 442 of the cord 440 can be configured to form lacing for the laced portion 430, for example lacing together the first edge 452 of the first elongate reinforcing portion 450 and the second edge 464 of the second elongate reinforcing portion 460.

A proximal portion 412 of the shaft 410 can be associated with, such as being coupled to, a distal end 406 of the hub 402. The shaft 410 can extend distally from the hub 402, such as from the distal end 406 of the hub 402. A hub delivery lumen 408 can extend through the hub 402, from a proximal end 404 to the distal end 406 of the hub 402. The shaft 410 can comprise a shaft delivery lumen 422 extending therethrough. The shaft delivery lumen 422 can be in fluid communication with the hub delivery lumen 408. For example, a medical device or delivery catheter carrying the medical device can be advanced through the hub delivery lumen 408, then into and through the shaft delivery lumen 422 from the proximal portion 412 to the distal portion 414 of the shaft 410. In some instances, the expandable shaft portion 420 can extend along an entire longitudinal dimension of the shaft 410, such as a dimension extending along a direction parallel or substantially parallel to a longitudinal axis of the shaft 410. For example, the expandable shaft portion 420 can extend along an entire longitudinal dimension of the shaft 410 extending distally from the hub 402. For example, the expandable shaft portion 420 can extend from a proximal end 416 to a distal end 418 of the shaft 410.

In some instances, the first and the second elongate reinforcing portion 450, 460 can each comprise a plurality of openings 456, 466 along a longitudinal dimension thereof. The cord 440 can be configured to be slidably disposed through the plurality of openings 456, 466 to form the lacing for the laced portion 430. Respective portions of the lacing portion 442 of the cord 440 can be slidably disposed through each of the openings 456, 466 to facilitate adjusting the distance between the first and second elongate reinforcing portions 450, 460. Adjusting the tension in the cord 440 can comprise sliding portions of the cord 440 through one or more of the openings 456, 466. First and second tail portions 444, 446 proximal of the lacing portion 442 can comprise a portion disposed through the hub delivery lumen 408. Portions of the first and second tail portions 444, 446 disposed proximally of the proximal end 404 of the hub 402 can be manipulated by an operator to adjust the tension in the cord 440.

The cord 440 is configured to be disposed over the externally oriented surface portion 494 of the first elongate foldable portion 492. For example, the first elongate foldable portion 492 can be coupled to respective portions of the first elongate reinforcing portion 450 and the second elongate reinforcing portion 460 such that the plurality of openings 456, 466 of the first and second elongate reinforcing portions 450, 460 can be disposed over the externally oriented surface portion 494.

The expandable access sheath 400 can have one or more other features of the expandable access sheath 200 described with reference to FIG. 2. For example, the expandable shaft portion 420 can comprise a second elongate foldable portion 470 extending along a second partial circumferential portion of the expandable shaft portion 420, and a third elongate foldable portion 490 extending along a third partial circumferential portion of the expandable shaft portion 420. The second and third partial circumferential portions can be non-overlapping with one another and non-overlapping with the first partial circumferential portion of the laced portion 430. The second elongate foldable portion 470 can extend between the second edge 454 of the first elongate reinforcing portion 450 and a first edge 482 of a third elongate reinforcing portion 480. The third elongate foldable portion 490 can between the first edge 462 of the second elongate reinforcing portion 460 and a second edge 484 of the third elongate reinforcing portion 480. For example, second elongate foldable portion 470 can extend from the second edge 454 of the first elongate reinforcing portion 450 to the first edge 482 of the third elongate reinforcing portion 480. The third elongate foldable portion 490 can extend from the first edge 462 of the second elongate reinforcing portion 460 to the second edge 484 of the third elongate reinforcing portion 480.

The expandable shaft portion 420 can assume increasingly more expanded state as the laced portion 430, and the second and third elongate foldable portions 470, 490 assume expanded and/or unfolded configurations. FIGS. 4A and 4B show the expandable shaft portion 420 in an expanded state. The laced portion 430 can be in an expanded configuration. The first elongate foldable portion 492 can be in an unfolded configuration. Each of the second and third elongate foldable portions 470, 490 can be in an unfolded configuration. The expandable shaft portion 420 can assume configurations with decreasing lateral cross-sectional size as the laced portion 430 assumes configurations having widths less than its width in its expanded configuration, and as the elongate foldable portions 470, 490 becoming increasingly folded. For example, while the expandable shaft portion 420 is in a collapsed state, the laced portion 430 can be in a collapsed configuration and both the first and second elongate foldable portions 470, 490 can be in a folded configuration. The lacing of the laced portion 430 can have slack or be in a tensioned state while the laced portion 430 is in the collapsed configuration. While the laced portion 430 is in the collapsed configuration, the first elongate foldable portion 492 can be in a folded configuration.

In some instances, one or more of the elongate foldable portions 470, 490, 492 can comprise an elasticity along a lateral dimension. For example, while the laced portion 430 is in a first expanded configuration, the first elongate foldable portion 492 can be in an unfolded configuration. While the laced portion 430 is in a second expanded configuration, the first elongate foldable portion 492 can be in an unfolded and stretched configuration. The lacing can comprise slack or be tensioned in the first and second expanded configurations. For example, the lacing, such as the lacing portion 442, can comprise slack while the laced portion 430 is in the first expanded configuration. The lacing, such as the lacing portion 442, can be tensioned while the laced portion is in the second expanded configuration.

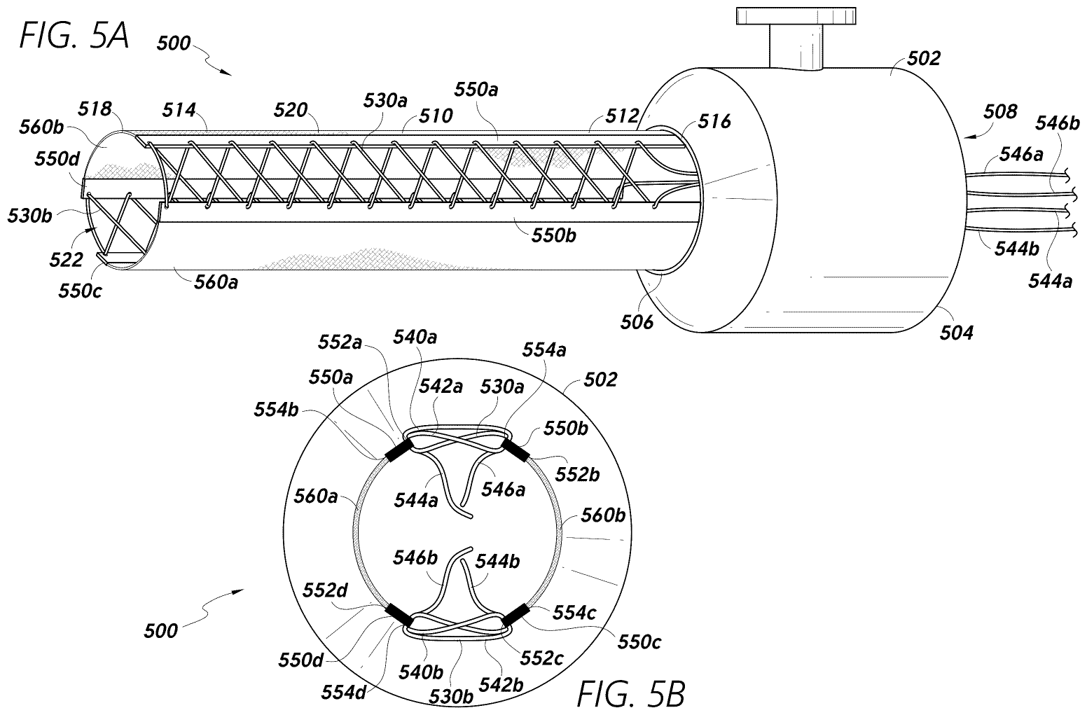

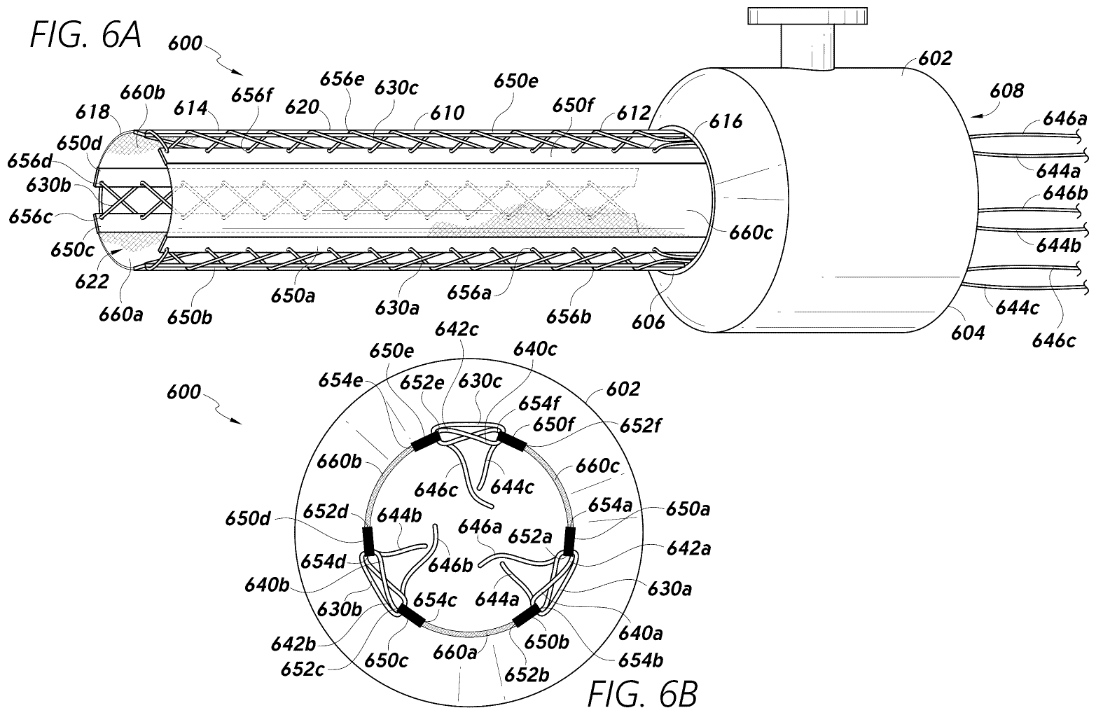

FIGS. 5A, 5B, 6A and 6B show examples of expandable access sheaths each comprising a plurality of laced portions. FIG. 5A is a perspective view, and FIG. 5B is a distal end view, of an example of an expandable access sheath 500 comprising an expandable shaft portion 520 that includes a first and a second laced portion 530a, 530b. The expandable access sheath 500 can comprise a shaft 510 comprising the expandable shaft portion 520. A proximal portion 512 of the shaft 510 can be associated with, such as being coupled to, a distal end 506 of a hub 502. The shaft 510 can extend distally from the hub 502, such as from the distal end 506 of the hub 502. A hub delivery lumen 508 can extend through the hub 502. The shaft 510 can comprise a shaft delivery lumen 522 extending therethrough. The shaft delivery lumen 522 can be in fluid communication with the hub delivery lumen 508. For example, a medical device can be advanced through the hub delivery lumen 508, then into and through the shaft delivery lumen 522 from the proximal portion 512 to the distal portion 514 of the shaft 510. The two laced portions 530a, 530b can be along opposingly oriented portions of the circumference of the expandable shaft portion 520. For example, the first and second laced portions 530a, 530b can be configured to extend around a first and a second circumferential portion of the expandable shaft portion 520, respectively. The first and second partial circumferential portions can be non-overlapping and form respective portions of the circumference of the expandable shaft portion 520. The first and second partial circumferential portions can have opposing orientations relative to one another around the circumference of the expandable shaft portion 520. In alternative instances, the first and second laced portions can extend around partial circumferential portions that are not opposingly oriented around the circumference. In some instances, the expandable shaft portion 520 can extend along an entire longitudinal dimension of the shaft 510, such as a dimension extending along a direction parallel or substantially parallel to a longitudinal axis of the shaft 510. For example, the expandable shaft portion 520 can extend along an entire longitudinal dimension of the shaft 510 extending distally from the hub 502, such as from a proximal end 516 to a distal end 518 of the shaft 510. The laced portions 530a, 530b can extend along an entire or substantially entire length of the expandable shaft portion 520.

Each of the laced portions 530a, 530b can comprise a cord 540a, 540b configured to form lacing. The first cord 540a of the first laced portion 530a can comprise at least a portion configured to form lacing extending between and to couple and/or lace together a first and a second elongate reinforcing portions 550a, 550b. Respective portions of the first cord 540a can pass through each of a plurality of openings extending along a longitudinal dimension of the first and second elongate reinforcing portions 550a, 550b. The second cord 540b of the second laced portion 530b can comprise at least a portion configured to form lacing extending between and to couple and/or lace together a third and a fourth elongate reinforcing portions 550c, 550d. Respective portions of the second cord 540b can slidably pass through each of a plurality of openings extending along a longitudinal dimension of the third and fourth elongate reinforcing portions 550c, 550d. Alternatively, respective portions of the cords 540a, 540b can slidably pass through each of a plurality of loops coupled to each of the elongate reinforcing portions 550a, 550b, 550c, 550d along a longitudinal dimension thereof. Cinching the lacing of the first laced portion 530a can reduce a distance between a first edge 552a of the first elongate reinforcing portion 550a and a second edge 554b of the second elongate reinforcing portion 550b. Cinching the lacing of the second laced portion 530b can reduce a distance between a first edge 552c of the third elongate reinforcing portion 550c and a second edge 554d of the fourth elongate reinforcing portion 550d. For example, a tension in the first cord 540a can be configured to be adjustable to change a distance between the first edge 552a of the first elongate reinforcing portions 550a and the second edge 554b of the second elongate reinforcing portion 550b. A tension in the second cord 540b can be configured to be adjustable to change a distance between the first edge 552c of the third elongate reinforcing portions 550c and the second edge 554d of the fourth elongate reinforcing portion 550d. Each of the first cord 540a and the second cord 540b can comprise a lacing portion 542a, 542b configured to form the lacing, and a first and a second tail portion 544a, 546a, 544b, 546b disposed proximally of the lacing portion 542a, 542b. An operator, such as a surgeon, can manipulate the proximally disposed portions of the cords 540a, 540b, such as the tail portion 544a, 546a, 544b, 546b to change the tension in the cords 540a, 540b. The first and second tail portion 544a, 546a, 544b, 546b can comprise a portion disposed through the hub delivery lumen 508 such that a portion of the first and second tail portion 544a, 546a, 544b, 546b can be disposed proximally of a proximal end 504 of the hub 502. The operator can cinch and/or pull on the cords 540a, 540b using the portions of the cords 540a, 540b disposed proximally of the hub 502.

In some instances, the expandable shaft portion 520 can comprise a first elongate foldable portion 560a and a second elongate foldable portion 560b extending around a third and fourth partial circumferential portion of the expandable shaft portion 520, respectively. The first elongate foldable portion 560a can extend between the second edge 554a of the first elongate reinforcing portion 550a and a first edge 552d of the fourth elongate reinforcing portion 550d. The second elongate foldable portion 560b can extend between a second edge 554c of the third elongate reinforcing portion 550c and the first edge 552b of the second elongate reinforcing portion 550b.

FIG. 6A is a perspective view, and FIG. 6B is a distal end view, of an example of an expandable access sheath 600 comprising an expandable shaft portion 620 that includes a first, second and third laced portion 630a, 630b, 630c. In some instances, the three laced portions 630a, 630b, 630c can be evenly distributed around a circumference of the expandable shaft portion 620. For example, the first, second and third laced portions 630a, 630b, 630c can be configured to extend around a first, a second and a third partial circumferential portion of the expandable shaft portion 620, respectively. The first, second and third partial circumferential portions can be non-overlapping and form respective portions of the circumference of the expandable shaft portion 620. The first, second, and third partial circumferential portions can be along partial circumferential portions that are evenly distributed around of the circumference of the expandable shaft portion 620. In alternative instances, the first, second and third partial circumferential portions may not be evenly distributed around the circumference of the expandable shaft portion 620. The expandable access sheath 600 can comprise a shaft 610 comprising the expandable shaft portion 620. A proximal portion 612 of the shaft 610 can be associated with, such as being coupled to, a distal end 606 of a hub 602. The shaft 610 can extend distally from the hub 602, such as from the distal end 606 of the hub 602. A hub delivery lumen 608 can extend through the hub 602. The shaft 610 can comprise a shaft delivery lumen 622 extending therethrough and be in fluid communication with the hub delivery lumen 608. For example, a medical device can be advanced through the hub delivery lumen 608, then into and through the shaft delivery lumen 622 from the proximal portion 612 to the distal portion 614 of the shaft 610. In some instances, the expandable shaft portion 620 can extend along an entire longitudinal dimension of the shaft 610, such as a dimension extending along a direction parallel or substantially parallel to a longitudinal axis of the shaft 610. For example, the expandable shaft portion 620 can extend along an entire longitudinal dimension of the shaft 610 extending distally from the hub 602, such as from a proximal end 616 to a distal end 618 of the shaft 610. The laced portions 630a, 630b, 630c can extend along an entire or substantially entire length of the expandable shaft portion 620.

Each of the laced portions 630a, 630b, 630c can comprise a cord 640a, 640b, 640c configured to form lacing configured to couple and/or lace together two elongate reinforcing portions. The first cord 640a of the first laced portion 630a can comprise a lacing portion 642a configured to form lacing extending between and to couple and/or lace together a first and a second elongate reinforcing portions 650a, 650b. Respective portions of the first cord 640a can slidably pass through each of a plurality of openings 656a, 656b extending along a longitudinal dimension of the first and second elongate reinforcing portions 650a, 650b. Adjusting the tension of the first cord 640a, such as by cinching and/or pulling on the first cord 640a, can reduce a distance between a first edge 652a of the first elongate reinforcing portion 650a and a second edge 654b of the second elongate reinforcing portion 650b. The second cord 640b can comprise a lacing portion 642b configured to form lacing extending between and to couple and/or lace together a third and a fourth elongate reinforcing portions 650c, 650d. Respective portions of the second cord 640b can slidably pass through each of a plurality of openings 656c, 656d extending along a longitudinal dimension of the third and fourth elongate reinforcing portions 650c, 650d. Adjusting the tension of the second cord 640b, such as by cinching and/or pulling on the second cord 640b, can reduce a distance between a first edge 652c of the third elongate reinforcing portion 650c and a second edge 654d of the fourth elongate reinforcing portion 650d. The third cord 640c can comprise a lacing portion 642c configured to form lacing extending between and to couple and/or lace together a fifth and a sixth elongate reinforcing portions 650c, 650f. Respective portions of the third cord 640c can slidably pass through each of a plurality of openings 656c, 656f extending along a longitudinal dimension of the fifth and sixth elongate reinforcing portions 650c, 650f. Adjusting the tension of the third cord 640c, such as by cinching and/or pulling on the third cord 640c, can reduce a distance between a first edge 652e of the fifth elongate reinforcing portion 650e and a second edge 654f of the sixth elongate reinforcing portion 650f. Alternatively, respective portions of the cords 640a, 640b, 640c can slidably pass through each of a plurality of loops coupled to each of the elongate reinforcing portions 650a, 650b, 650c, 650d, 650c, 650f along a longitudinal dimension thereof.

Each of the cords 640a, 640b, 640c can comprise a first and a second tail portion 644a, 646a, 644b, 646b, 644c, 646c disposed proximally of the lacing portion 642a, 642b, 642c. The first and second tail portions 644a, 646a, 644b, 646b, 644c, 646c can comprise a portion disposed through the hub delivery lumen 608 such that a portion of the first and second tail portions 644a, 646a, 644b, 646b, 644c, 646c can be disposed proximally of a proximal end 604 of the hub 602. An operator, such as a surgeon, can cinch and/or pull on the cords 640a, 640b, 640c using the portions of the cords 640a, 640b, 640c disposed proximally of the hub 602.

The expandable shaft portion 620 can comprise a first elongate foldable portion 660a, a second elongate foldable portion 660b, and a third elongate foldable portion 660c extending around a fourth, fifth and sixth partial circumferential portion of the expandable shaft portion 620, respectively. Each of first, second, third, fourth, fifth and sixth partial circumferential portions can be non-overlapping with one another. The first, second, third, fourth, fifth and sixth partial circumferential portions can each form respective portions of the circumference of the expandable shaft portion 620. The first elongate foldable portion 660a can extend between the first edge 652b of the second elongate reinforcing portion 650b and a second edge 654c of the third elongate reinforcing portion 650c. For example, the first elongate foldable portion 660a can extend from the first edge 652b of the second elongate reinforcing portion 650b to the second edge 654c of the third elongate reinforcing portion 650c. The second elongate foldable portion 660b can extend between a first edge 652d of the fourth elongate reinforcing portion 650d and a second edge 654c of the fifth elongate reinforcing portion 650c. For example, the second elongate foldable portion 660b can extend from the first edge 652d of the fourth elongate reinforcing portion 650d to the second edge 654c of the fifth elongate reinforcing portion 650c. The third elongate foldable portion 660c can extend between a first edge 652f of the sixth elongate reinforcing portion 650f and the second edge 654a of the first elongate reinforcing portion 650a. For example, the third elongate foldable portion 660c can extend from the first edge 652f of the sixth elongate reinforcing portion 650f to the second edge 654a of the first elongate reinforcing portion 650a.

FIGS. 5A and 5B show the expandable shaft portion 520 in an expanded state. The laced portions 530a, 530b can each be in an expanded configuration. Each of the first and second elongate foldable portions 560a, 560b can be in an unfolded configuration. FIGS. 6A and 6B show the expandable shaft portion 620 in an expanded state. The laced portions 630a, 630b, 630c can be in expanded configurations. Each of the first, second and third elongate foldable portions 660a, 660b, 660c can be in an unfolded configurations. As described herein, elongate foldable portions can be increasingly folded or unfolded, and the laced portions can assume configurations of various width, to provide a corresponding decrease or increase in a lateral cross-sectional size of the expandable access sheath. In some instances, one or more of the elongate foldable portions can comprise an elasticity along a lateral dimension such that an elongate foldable portion can be both unfolded and stretched. The lateral dimension can be perpendicular or substantially perpendicular to the longitudinal axis of the respective shaft.

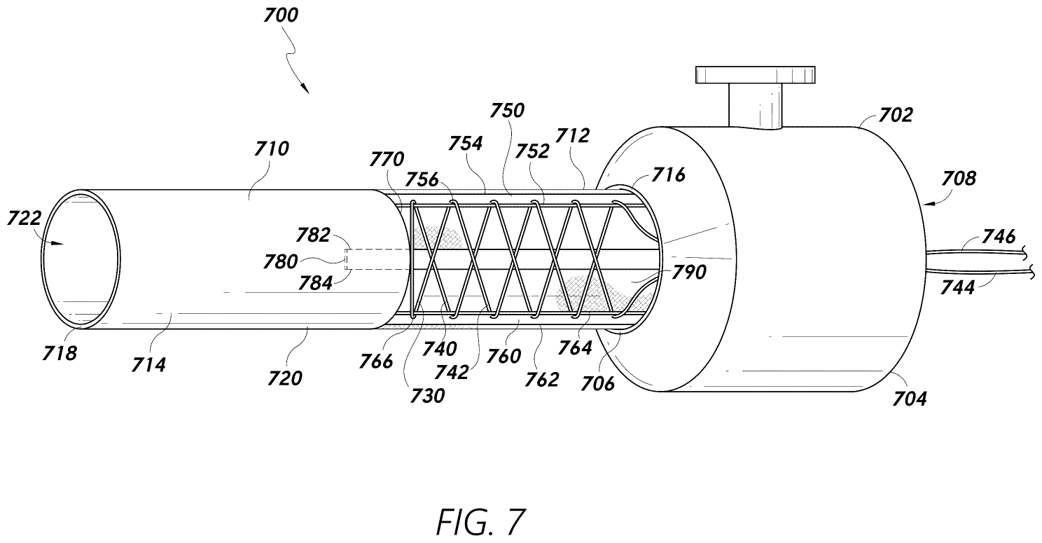

FIG. 7 is a perspective view of an example of an expandable access sheath 700 comprising an expandable shaft portion 720 on a proximal portion 712 of its shaft 710. A circumference of the expandable shaft portion 720 can comprise a laced portion, a plurality of elongate foldable portions, and a plurality of elongate reinforcing portions, each of the laced portion, plurality of elongate foldable portions, and plurality of elongate reinforcing portions extending along a corresponding portion of the circumference that do not overlap with one another. In some instances, the expandable shaft portion 720 can extend along a proximal half of the shaft 710. A proximal portion 712, such as a proximal half, of the shaft 710 is the expandable shaft portion 720. The proximal half can be a proximal half of the shaft 710 extending distally from a distal end 706 of the hub 702, such as a proximal half between a proximal end 716 and a distal end 718 of the shaft 710. A distal portion 714 of the shaft 710 may not be an expandable shaft portion.

The proximal portion 712 of the shaft 710 can be associated with, such as being coupled to, the distal end 706 of a hub 702. A hub delivery lumen 708 can extend through the hub 702, from a proximal end 704 to the distal end 706 of the hub 702. The shaft 710 can extend distally from the hub 702, such as from the distal end 706 of the hub 702. The shaft delivery lumen 722 can be in fluid communication with the hub delivery lumen 708. A sheath delivery lumen extending through the expandable access sheath 700 can comprise the hub delivery lumen 708 and the shaft delivery lumen 722. In some instances, the expandable shaft portion 720 can extend along a proximal portion 712 of the shaft 710 that is distal of the distal end 706 of the hub 702. In some instances, the expandable shaft portion 720 can be a proximal half of the shaft 710 that is distal of the distal end 706 of the hub 702.

Referring to FIG. 7, a laced portion 730 can extend around a first partial circumferential portion of the expandable shaft portion 720. A longitudinal dimension of the laced portion 730, such as a length extending along a direction parallel or substantially parallel to a longitudinal axis of the shaft 710, can be along a proximal portion 712 of the shaft 710. A lateral dimension, such as a dimension extending around a circumference of the shaft 710, of the laced portion 730 can be along the first partial circumferential portion. The laced portion 730 can comprise one or more features of the laced portion 230 described with reference to the expandable access sheath 200 described with reference to FIG. 2. For example, the laced portion 730 can comprise a cord 740 configured to form lacing for the laced portion 730 and to lace together a first and a second elongate reinforcing portion 750, 760. The first and second elongate reinforcing portions 750 can extend along the proximal portion 712 of the shaft 710. The cord 740 can comprise a lacing portion 742 configured to form the lacing, and a first and a second tail portion 744, 746 disposed proximally of the lacing portion 742. The laced portion 730 can extend between the first and second elongate reinforcing portions 750, 760. For example, respective portions of the cord 740 can slidably pass through each of a plurality of openings 756, 766 extending along the first and second elongate reinforcing portion 750, 760. Alternatively, respective portions of the cord 740 can pass through each of a plurality of loops coupled to each of the first edge 752 of the first elongate reinforcing portion 750 and the second edge 764 of the second elongate reinforcing portion 760. Cinching the lacing can reduce a distance between a first edge 752 of the first elongate reinforcing portion 750 and a second edge 764 of the second elongate reinforcing portion 760. A tension in the cord 740 can be configured to be adjustable to change a distance between the first and the second elongate reinforcing portions 750, 760, such as between the first edge 752 and the second edge 764. The first and second tail portions 744, 746 can comprise a portion extending through the hub delivery lumen 708 such that a portion of each of the first and second tail portions 744, 746 disposed proximally of the proximal end 704 of the hub 702 can be manipulated by the operator.

In some instances, the expandable shaft portion 720 can comprise a first elongate foldable portion 770 and a second elongate foldable portion 790 extending around a second and third partial circumferential portion of the expandable shaft portion 720, respectively. The first elongate foldable portion 770 can extend between the first elongate reinforcing portion 750 and a third elongate reinforcing portion 780, such as between a second edge 754 of the first elongate reinforcing portion 750 and a first edge 782 of a third elongate reinforcing portion 780. The second elongate foldable portion 790 can extend between a second edge 784 of the third elongate reinforcing portion 780 and the first edge 762 of the second elongate reinforcing portion 760. The expandable shaft portion 720 is shown in an expanded state in FIG. 7. For example, the laced portion 730 is shown in an expanded configuration, and the first and second elongate foldable portions 770, 790 are shown in unfolded configurations.

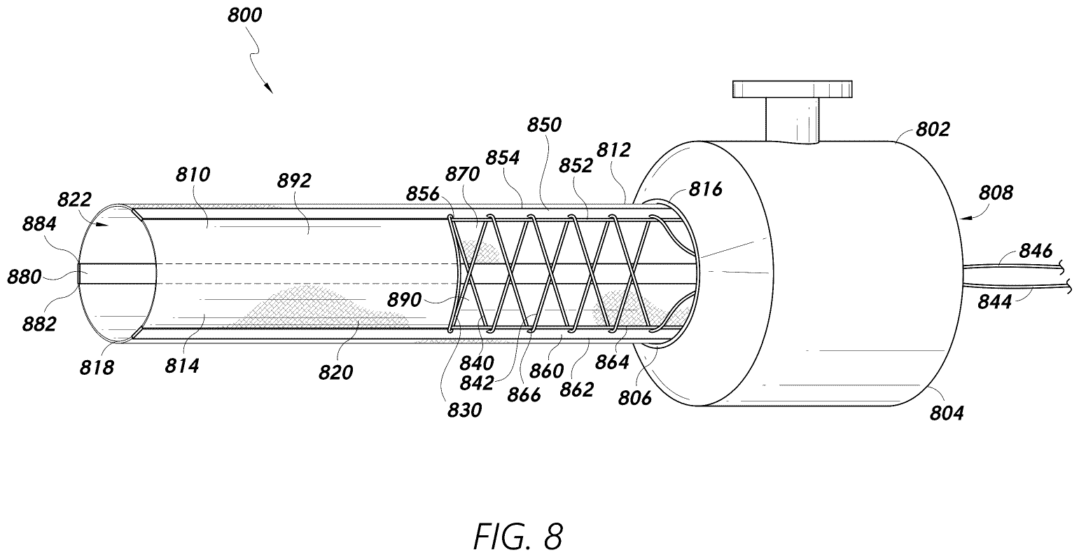

FIG. 8 is a perspective view of example of an expandable access sheath 800 comprising a shaft 810 that includes an expandable shaft portion 820 with a laced portion 830 extending along a portion of a longitudinal dimension of the expandable shaft portion 820. The longitudinal dimension can be a length, for example being parallel or substantially parallel to a longitudinal axis of the shaft 810. In some instances, the laced portion 830 is on a proximal portion 812 of the expandable shaft portion 820. A proximal portion 812 of the shaft 810 can be associated with, such as being coupled to, a distal end 806 of a hub 802. A hub delivery lumen 808 can extend through the hub 802. At least a portion of the shaft 810 can extend distally from the hub 802, such as from the distal end 806 of the hub 802. The shaft delivery lumen 822 can be in fluid communication with the hub delivery lumen 808. In some instances, the laced portion 830 is on a proximal half of the expandable shaft portion 820. In some instances, the expandable shaft portion 820 can extend along an entire length of the shaft 810. In some instances, the expandable shaft portion 820 can extend along an entire length of the shaft 810 disposed distally of the distal end 806 of the hub 802, such as from a proximal end 816 to a distal end 818 of the shaft 810.

A proximal portion 812, such as a proximal half, of the expandable shaft portion 820 can comprise a laced portion 830 extending around a first partial circumferential portion of the expandable shaft portion 820. A longitudinal dimension of the laced portion 830 can be along a proximal portion 812 of the expandable shaft portion 820. A lateral dimension, such as a dimension extending around a circumference of the expandable shaft portion 820, of the laced portion 830 can be along the first partial circumferential portion. The laced portion 830 can extend between a first and a second elongate reinforcing portion 850, 860. The laced portion 830 can comprise a cord 840 comprising a lacing portion 842 configured to form lacing for the laced portion 830 and to lace together the first and second elongate reinforcing portions 850, 860. Respective portions of the cord 840 can pass through each of a plurality of openings 856, 866 extending along the first and second elongate reinforcing portions 850, 860. Alternatively, respective portions of the cord 840 can pass through each of a plurality of loops coupled to each of the first edge 852 of the first elongate reinforcing portion 850 and the second edge 864 of the second elongate reinforcing portion 860. The cord 840 can comprise a first and a second tail portion 844, 846 configured to be disposed proximally of the lacing portion 842. Cinching the lacing, such as by adjusting tension in the cord 840, can reduce a distance between a first edge 852 of the first elongate reinforcing portion 850 and a second edge 864 of the second elongate reinforcing portion 860. An operator can manipulate portions of the first and second tail portions 844, 846 disposed proximally of a proximal end 804 of the hub 802 to adjust the tension in the cord 840.

The first and second elongate reinforcing portions 850, 860 can extend along an entire length of the expandable shaft portion 820, such as a length of the expandable shaft portion 820 distal of the distal end 806 of the hub 802. A distal portion 814 of the shaft 810 can comprise a third elongate foldable portion 892 extending between the first edge 852 of the first elongate reinforcing portion 850 and the second edge 864 of the second elongate reinforcing portion 860. The expandable shaft portion 820 can comprise a second elongate foldable portion 870 and a third elongate foldable portion 890 extending around a second and third partial circumferential portion of the expandable shaft portion 820, respectively. The second elongate foldable portion 870 can extend between the first elongate reinforcing portion 850 and a third elongate reinforcing portion 880, such as between a second edge 854 of the first elongate reinforcing portion 850 and a first edge 882 of the third elongate reinforcing portion 880. The third elongate foldable portion 880 can extend between a second edge 884 of the third elongate reinforcing portion 880 and the first edge 862 of the second elongate reinforcing portion 860. The second and third elongate foldable portions 870, 890 can extend along an entire length of the expandable shaft portion 820. FIG. 8 shows the expandable access sheath 800 in an expanded state, for example the laced portion 830 in an expanded configuration and the elongate foldable portions 870, 890, 892 in unfolded configurations.

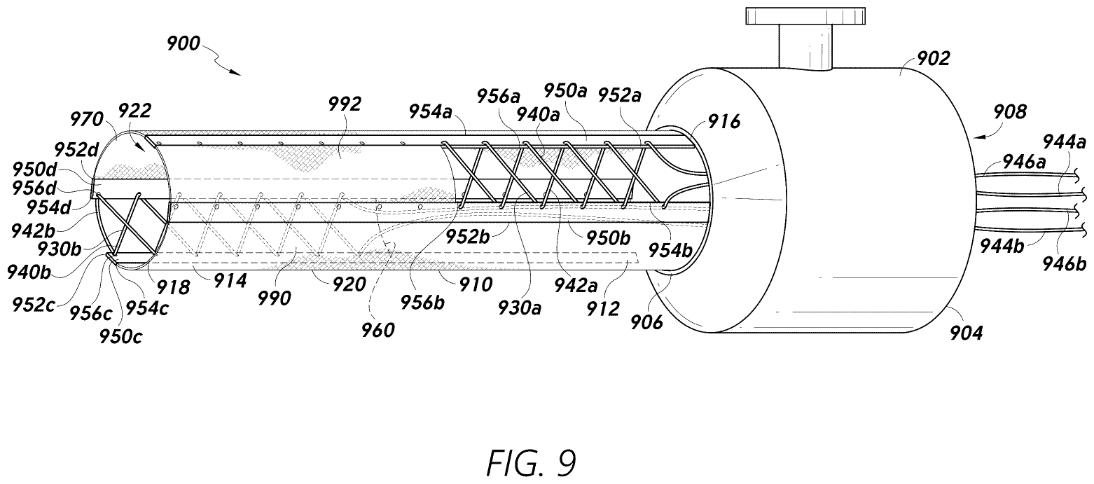

FIG. 9 is a perspective view of an example of an expandable access sheath 900 comprising a shaft 910 that includes an expandable shaft portion 920 with a first laced portion 930a extending around a first partial circumferential portion along a proximal portion 912 of the shaft 910 and a second laced portion 930b extending around a second partial circumferential portion along a distal portion 914 of the shaft 910. The proximal portion 912 of the shaft 910 can be associated with, such as being coupled to, a distal end 906 of a hub 902. At least a portion of the shaft 910 can extend distally from the hub 902, such as from the distal end 906 of the hub 902. A hub delivery lumen 908 can extend through the hub 902 and be in fluid communication with a shaft delivery lumen 922 extending through the shaft 910. In some instances, the first laced portion 930a can extend along a proximal half of the shaft 910. In some instances, the second laced portion 930b can extend along a distal half of the shaft 910. The first and second partial circumferential portions can be opposingly oriented around the circumference of the shaft 910. For example, the first and second laced portions 930a, 930b can be opposingly oriented around the circumference of the shaft 910. In alternative instances, the first and second partial circumferential portions may not be opposingly oriented around the circumference of the shaft 910.

The first laced portion 930a can comprise a first cord 940a comprising a lacing portion 942a configured to form lacing extending between and to couple and/or lace together a first and a second elongate reinforcing portions 950a, 950b. Respective portions of the first cord 940a can pass through each of a plurality of openings 956a, 956b extending along the first and second elongate reinforcing portions 950a, 950b. The second cord 940b of the second laced portion 930b can comprise a lacing portion 942b configured to form lacing extending between and to couple and/or lace together a third and a fourth elongate reinforcing portions 950c, 950d. Respective portions of the second cord 940b can pass through each of a plurality of openings 956c, 956d extending along the third and fourth elongate reinforcing portions 950c, 950d. Alternatively, respective portions of the cords 940a, 940b can pass through each of a plurality of loops coupled to each of the elongate reinforcing portions 950a, 950b, 950c, 950d. Cinching the lacing of the first laced portion 930a can reduce a distance between corresponding portions of a first edge 952a of the first elongate reinforcing portion 950a and a second edge 954b of the second elongate reinforcing portion 950b. Cinching the lacing of the second laced portion 930b can reduce a distance between a first edge 952c of the third elongate reinforcing portion 950c and a second edge 954d of the fourth elongate reinforcing portion 950d. For example, a tension in the first cord 940a can be configured to be adjustable to change a distance between corresponding portions of the first edge 952a of the first elongate reinforcing portion 950a and a second edge 954b of the second elongate reinforcing portion 950b. A tension in the second cord 940b can be configured to be adjustable to change a distance between corresponding portions of the first edge 952c of the third elongate reinforcing portion 950c and the second edge 954d of the fourth elongate reinforcing portion 950d. Each of the first cord 940a and the second cord 940b can comprise a first and a second tail portion 944a, 946a, 944b, 946b disposed proximally of the lacing portion 942a, 942b. The proximally disposed portions of the cords 940a, 940b, such as the tail portion 944a, 946a, 944b, 946b, can extend through the hub delivery lumen 908 such that a portion disposed proximally of the proximal end 904 of the hub 902 can be manipulated by an operator, such as a surgeon, to change the tension in the cords 940a, 940b.

The expandable shaft portion 920 can comprise a first elongate foldable portion 992 extending along the distal portion of the shaft 910 around the first partial circumferential portion. The first elongate foldable portion 992 can extend between the first edge 952a of the first elongate reinforcing portion 950a and the second edge 954b of the second elongate reinforcing portion 950b. A second elongate foldable portion 970 can extend along a proximal portion 912 of the shaft 910 along the second partial circumferential portion of the shaft 910. The second elongate foldable portion 960 can extend between the first edge 952c of the third elongate reinforcing portion 950c and the second edge 954d of the fourth elongate reinforcing portion 950d. The expandable shaft portion 920 can comprise a third elongate foldable portion 970 extending along a third partial circumferential portion and a fourth elongate foldable portion 990 extending along a fourth partial circumferential portion. The first, second third and fourth partial circumferential portions can be non-overlapping portions. The first, second third and fourth partial circumferential portions can form respective portions of the circumference of the expandable shaft portion 920. The third and fourth elongate foldable portions 970, 990 can extend along an entire length of the shaft 910, including an entire length of the shaft 910 disposed distally of the hub 202. For example, the third and fourth elongate foldable portions 970, 990 can extend from a proximal end 916 to a distal end 918 of the shaft 910. The third elongate foldable portion 970 can extend between a first edge 952b of the second elongate reinforcing portion 950b and a second edge 954c of the third elongate reinforcing portion 950c. The fourth elongate foldable portion 990 can extend between a first edge 952d of the fourth elongate reinforcing portion 950d and a second edge 954a of the first elongate reinforcing portion 950a. For example, the first elongate foldable portion 992 can extend from the first edge 952a of the first elongate reinforcing portion 950a to the second edge 954b of the second elongate reinforcing portion 950b. The second elongate foldable portion 960 can extend from the first edge 952c of the third elongate reinforcing portion 950c to the second edge 954d of the fourth elongate reinforcing portion 950d. The third elongate foldable portion 970 can extend from the first edge 952b of the second elongate reinforcing portion 950b to the second edge 954c of the third elongate reinforcing portion 950c. The fourth elongate foldable portion 990 can extend from the first edge 952d of the fourth elongate reinforcing portion 950d to the second edge 954a of the first elongate reinforcing portion 950a. FIG. 9 shows the expandable access sheath 900 in an expanded state, for example the laced portions 930a, 930b in an expanded configuration and the elongate foldable portions 960, 970, 990, 992 in unfolded configurations.

It will be understood that the expandable access sheaths 700, 800, 900 described with reference to FIGS. 7,8 and 9 can assume less expanded states, and/or a collapsed state, as described herein. Elongate foldable portions can be increasingly folded, and/or the laced portions can assume configurations of various widths, to provide a corresponding decrease in a lateral cross-sectional size of the expandable access sheaths 700, 800, 900. In some instances, one or more of the elongate foldable portions can have an elasticity along a lateral dimension such that an elongate foldable portion can be both unfolded and stretched. The lateral dimension can be perpendicular or substantially perpendicular to the longitudinal axis of the respective shaft.

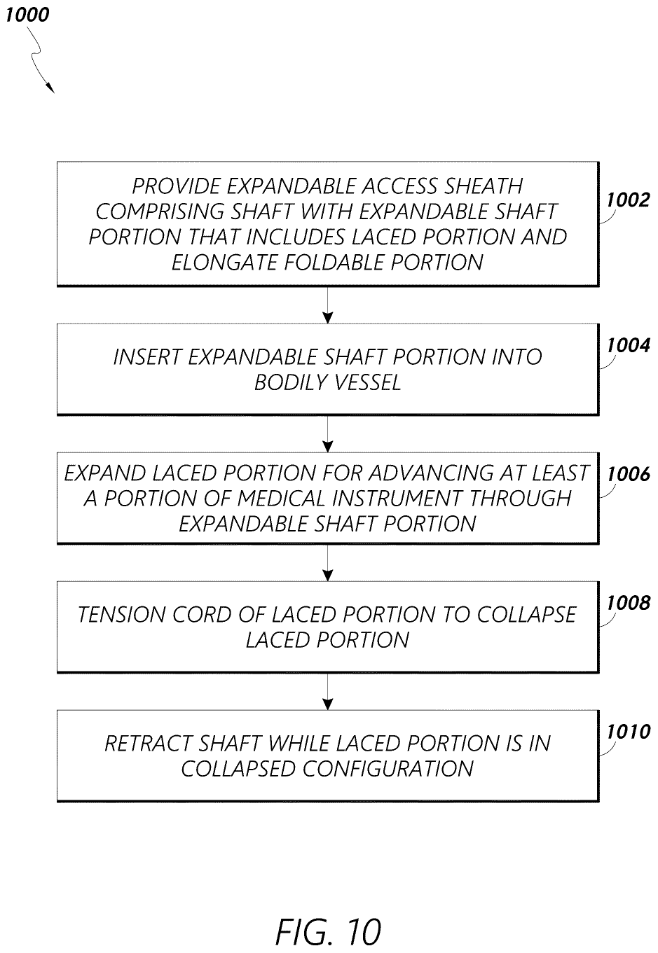

FIG. 10 is a process flow diagram illustrating an example of a delivery process 1000 for delivering a medical device using an expandable access sheath having one or more features described herein. In block 1002, the process can involve providing an expandable access sheath. The expandable access sheath can comprise a hub, and a shaft extending distally from the hub, the shaft comprising an expandable shaft portion. The expandable shaft portion can comprise a first elongate foldable portion extending along a first partial circumferential portion of the expandable shaft portion, and a laced portion extending along a second partial circumferential portion of the expandable shaft portion. The laced portion can comprise a cord configured to form lacing for the laced portion. In block 1004, the process can involve inserting the expandable shaft portion into a bodily lumen. For example, at least a portion of the shaft, including the expandable shaft portion, can be advanced into the bodily lumen to a target position within the bodily lumen. After the expandable shaft portion is desirably positioned within the bodily lumen, a medical instrument can be advanced through the shaft delivery lumen. The expandable shaft portion can be inserted into any number of lumens and/or vessels of a patient to facilitate delivery of medical devices to any number of organs, chambers, vessels and/or lumens. For example, the process can involve inserting at least a portion of the expandable shaft portion into a femoral artery for delivery of medical devices to a heart. In some instances, another arterial access can be used. In some instances, a venous access approach can be used, such as a femoral vein, internal jugular vein, and/or subclavian vein.

In block 1006, the process can involve expanding the laced portion for advancing at least a portion of a medical instrument through the expandable shaft portion. For example, the laced portion can be expanded for advancing at least a portion of a medical instrument through a corresponding portion of the shaft delivery lumen extending through the expandable shaft portion.

In block 1008, the process can involve tensioning the cord to collapse the laced portion. The expandable shaft portion can be configured to be transformed into a reduced profile configuration to facilitate its withdrawal through the anatomical pathway. For example, prior to withdrawal of the shaft from the bodily lumen, the laced portion can be transformed to a collapsed configuration. In block 1010, the process can involve retracting the shaft while the laced portion is in the collapsed configuration.

In some instances, the laced portion can comprise lacing coupling adjacent elongate reinforcing portions such that expanding the laced portion can comprise pushing apart the adjacent elongate reinforcing portions. In some instances, expanding the laced portion can comprise adjusting tension in the cord to allow pushing apart of the adjacent elongate reinforcing portions. For example, tension in the cord can be adjusted to allow slack in the lacing, thereby allowing pushing apart of the adjacent elongate reinforcing portions. Reducing tension in the cord can provide slack in the cord such that adjacent elongate reinforcing portions can be pushed apart to enlarge a lateral cross-sectional size of the expandable shaft portion. In some instances, expanding the laced portion for advancing at least a portion of a medical instrument through the expandable shaft portion can comprise expanding the laced portion for advancing a medical device through the expandable shaft portion.

In some instances, the laced portion can comprise a second elongate foldable portion. The cord can be disposed over an externally oriented surface of the second elongate foldable portion. For example, expanding the laced portion can comprise unfolding the second elongate foldable portion. In some instances, expanding the laced portion can comprise unfolding and stretching the second elongate foldable portion. In some instances, collapsing the laced portion can comprise folding the second elongate foldable portion.

In some instances, the first elongate foldable portion can be unfolded for advancing the at least a portion of the medical instrument through the corresponding portion of the shaft delivery lumen. In some instances, the first elongate foldable portion can be unfolded and stretched for advancing the at least a portion of the medical instrument through the corresponding portion of the shaft delivery lumen.

In some instances, tensioning the cord can comprise proximally translating proximally disposed portions of the cord. In some instances, tensioning the cord can comprise proximally translating portions of the cord disposed proximally of a proximal end of the hub. For example, proximally disposed portions of the cord can comprise cord tail portions. An operator, such as a surgeon, can pull on the proximally disposed portions, including the cord tail portions, to proximally translated portions of the cord to tension the lacing. In some instances, collapsing the laced portion can comprise proximally translating the cord tail portions to reduce a distance between adjacent elongate reinforcing portions coupled to one another by the cord. In some instances, collapsing the laced portion can comprise tensioning the cord to position the adjacent elongate reinforcing portions in contact with one another.

In some instances, inserting the expandable shaft portion can comprises inserting the expandable shaft portion comprising an introducer preloaded therethrough. For example, an introducer can be preloaded within an expandable access sheath while at least a portion of the expandable shaft portion is advanced to a target position within an anatomical pathway. The introducer can be positioned at least partially within the shaft delivery lumen. While preloaded within the shaft delivery lumen, a distal portion of the introducer, including a distal end of the introducer, can be disposed distally of a distal end of the shaft. In some instances, a distal end of the expandable shaft portion can be the distal end of the shaft. The distal end of the introducer can be distal of the distal end of the expandable shaft portion.

In some instances, inserting the expandable shaft portion comprising the introducer preloaded therethrough can comprise maintaining slack in the cord of the laced portion while inserting the expandable shaft portion. In some instances, the laced portion can be maintained in the collapsed configuration while inserting the expandable shaft portion with the introducer preloaded. For example, the laced portion can be collapsed and the lacing can have slack. In some instances, inserting the expandable shaft portion comprising the introducer preloaded therethrough can comprise maintaining the laced portion in the collapsed configuration and the cord of the laced portion in a tensioned state while inserting the expandable shaft portion. In some instances, inserting the expandable shaft portion comprising the introducer preloaded therethrough can comprise maintaining the cord of the laced portion in an expanded configuration while inserting the expandable shaft portion. For example, the laced portion can be in an expanded configuration and the lacing can be in a tensioned state while the introducer is preloaded within the expandable shaft portion. In some instances, inserting the expandable shaft portion comprising the introducer preloaded therethrough can comprise inserting the expandable shaft portion while the first elongate foldable portion is unfolded, unfolded and stretched, or folded.

### ADDITIONAL DESCRIPTION OF EXAMPLES

Provided below is a list of examples, each of which may include aspects of any of the other examples disclosed herein. Furthermore, aspects of any example described above may be implemented in any of the numbered examples provided below.
Example 1: An expandable access sheath comprising a hub having a hub delivery lumen extending therethrough, and a shaft extending distally from the hub and comprising a shaft delivery lumen extending therethrough and in fluid communication with the hub delivery lumen. The shaft can comprise an expandable shaft portion having a laced portion extending along a first partial circumferential portion of the expandable shaft portion, the laced portion comprising a cord configured to form lacing for the laced portion, and an elongate foldable portion extending along a second partial circumferential portion of the expandable shaft portion.
Example 2: The sheath of any example herein, in particular example 1, wherein a tension in the cord of the laced portion is configured to be adjustable to change a width of the laced portion.
Example 3: The sheath of any example herein, in particular example 2, wherein proximally extending portions of the cord are configured to be disposed proximally of a proximal end of the hub, tension in the proximally extending portions being configured to be adjustable to adjust a tension in the cord.
Example 4: The sheath of any example herein, in particular examples 1 to 3, wherein the laced portion is in an expanded configuration and the elongate foldable portion is in a folded configuration while the expandable shaft portion is in a first expanded state, the laced portion is in an expanded configuration and the elongate foldable portion is in an unfolded configuration while the expandable shaft portion is in a second expanded state, and the laced portion is in a collapsed configuration and the elongate foldable portion is in a folded configuration while the expandable shaft portion is in a collapsed state.
Example 5: The sheath of any example herein, in particular example 4, wherein the lacing of the laced portion comprises slack while the laced portion is in the collapsed configuration.
Example 6: The sheath of any example herein, in particular example 4, wherein the lacing of the laced portion is in a tensioned state while the laced portion is in the collapsed configuration.
Example 7: The sheath of any example herein, in particular examples 1 to 6, wherein the elongate foldable portion comprises elasticity along a lateral dimension.
Example 8: The sheath of any example herein, in particular example 7, wherein: the laced portion is in an expanded configuration and the elongate foldable portion is in a folded configuration, while the expandable shaft portion is in a first expanded state; the laced portion is in the expanded configuration and the elongate foldable portion is in an unfolded configuration, while the expandable shaft portion is in a second expanded state; the laced portion is in the expanded configuration and the elongate foldable portion is in an unfolded and stretched configuration while the expandable shaft portion is in a third expanded state; and the laced portion in a collapsed configuration and the elongate foldable portion is in a folded configuration while the expandable shaft portion is in a collapsed state.
Example 9: The sheath of any example herein, in particular example 8, wherein the lacing of the laced portion comprises slack while the laced portion is in the collapsed configuration.
Example 10: The sheath of any example herein, in particular example 8, wherein the lacing of the laced portion is in a tensioned state while the laced portion is in the collapsed configuration.
Example 11: The sheath of any example herein, in particular examples 1 to 10, wherein the laced portion further comprises a second elongate foldable portion along the first partial circumferential portion of the expandable shaft portion, the cord being disposed over an externally oriented surface of the second elongate foldable portion.
Example 12: The sheath of any example herein, in particular example 11, wherein the second elongate foldable portion comprises elasticity along a lateral dimension.
Example 13: The sheath of any example herein, in particular example 12, wherein the laced portion is in a first expanded configuration and the elongate foldable portion is in a folded configuration, while the expandable shaft portion is in a first expanded state, the lacing comprising slack and the second elongate foldable portion in an unfolded configuration while the laced portion is in the first expanded state. The laced portion can be in a second expanded configuration and the elongate foldable portion can be in a folded configuration, while the expandable shaft portion is in a second expanded state, the lacing being tensioned and the second elongate foldable portion in an unfolded and stretched configuration while the laced portion is in the second expanded state. The laced portion can be in the second expanded configuration and the elongate foldable portion can be in an unfolded configuration, while the expandable shaft portion is in a third expanded state. The laced portion can be in a collapsed configuration and the elongate foldable portion can be in a folded configuration while the expandable shaft portion is in a collapsed state.
Example 14: The sheath of any example herein, in particular example 13, wherein the lacing of the laced portion is in a tensioned state while the laced portion is in the collapsed configuration.
Example 15: The sheath of any example herein, in particular example 13, wherein the elongate foldable portion comprises elasticity along a lateral dimension.
Example 16: The sheath of any example herein, in particular examples 11 to 15, wherein the elongate foldable portion comprises elasticity along a lateral dimension.
Example 17: The sheath of any example herein, in particular example 16, wherein the laced portion is in a first expanded configuration and the elongate foldable portion is in a folded configuration, while the expandable shaft portion is in a first expanded state, the lacing comprising slack and the second elongate foldable portion in an unfolded configuration while the laced portion is in the first expanded state. The laced portion can be in a second expanded configuration and the elongate foldable portion can be in a folded configuration, while the expandable shaft portion is in a second expanded state, the lacing being tensioned and the second elongate foldable portion in an unfolded and stretched configuration while the laced portion is in the second expanded state. The laced portion can be in the second expanded configuration and the elongate foldable portion can be in an unfolded configuration, while the expandable shaft portion is in a third expanded state. The laced portion can be in the second expanded configuration and the elongate foldable portion can be in an unfolded and stretched configuration, while the expandable shaft portion is in a fourth expanded state. The laced portion can be in a collapsed configuration and the elongate foldable portion can be in a folded configuration while the expandable shaft portion is in a collapsed state.
Example 18: The sheath of any example herein, in particular example 17, wherein the lacing of the laced portion is in a tensioned state while the laced portion is in the collapsed configuration.
Example 19: The sheath of any example herein, in particular example 17, wherein the elongate foldable portion comprises elasticity along a lateral dimension.
Example 20: The sheath of any example herein, in particular examples 1 to 19, further comprising a first and a second elongate reinforcing portion and wherein the laced portion comprises a cord lacing together a first edge of the first elongate reinforcing portion and a second edge of the second elongate reinforcing portion, the first edge and the second edge being configured to be oriented toward one another.
Example 21: The sheath of any example herein, in particular example 20, wherein a tension in the cord of the laced portion is configured to be adjustable to change a distance between the first and the second elongate reinforcing portions.
Example 22: The sheath of any example herein, in particular example 20 or 21, wherein the first and a second elongate reinforcing portion each comprise a plurality of openings along a longitudinal dimension, the cord of the laced portion being configured to be passed through the plurality of openings to form the lacing.
Example 23: The sheath of any example herein, in particular example 20 or 21, wherein a plurality of loops is coupled to each of the first edge of the first elongate reinforcing portion and the second edge of the second elongate reinforcing portion, the cord of the laced portion being configured to be passed through the plurality of loops to form the lacing.
Example 24: The sheath any example herein, in particular examples 1 to 23, wherein the expandable shaft portion is along a proximal portion of shaft.
Example 25: The sheath of any example herein, in particular example 24, wherein the expandable shaft portion is along proximal half of shaft.
Example 26: The sheath any example herein, in particular examples 1 to 23, wherein the expandable shaft portion is along an entire length of the shaft disposed distally of the hub.
Example 27: The sheath of any example herein, in particular example 26, wherein the laced portion is along an entire length of the shaft disposed distally of the hub.
Example 28: The sheath of any example herein, in particular examples 1 to 26, wherein the laced portion is along a proximal portion of shaft.
Example 29: The sheath of any example herein, in particular example 28, wherein the laced portion is along proximal half of shaft.
Example 30: The sheath any example herein, in particular examples 1 to 23, wherein the laced portion extending around the first partial circumferential portion of the expandable shaft portion is along a first longitudinal dimension of the expandable shaft portion, and the expandable shaft portion further comprises a second laced portion extending around a third partial circumferential portion of the expandable shaft portion, the second laced portion being along a second longitudinal dimension.
Example 31: The sheath of any example herein, in particular examples 1 to 23 wherein the expandable shaft portion comprises: a plurality of elongate reinforcing portions and at least one elongate foldable portion, each of the plurality of elongate reinforcing portions and at least one elongate foldable portion extending around respective portions of a circumference of the expandable shaft portion; and a plurality of laced portions evenly distributed around and extending around respective remaining portions of the circumference of the expandable shaft portion, each laced portion coupling a respective pair of elongate reinforcing portions.
Example 32: The sheath of any example herein, in particular example 31, wherein the laced portion further comprises an elongate foldable portion, and a cord of the laced portion is disposed over an externally oriented surface of an elongate foldable portion.
Example 33: The sheath of any example herein, in particular example 32, wherein the elongate foldable portion of the laced portion comprises elasticity along a lateral dimension.
Example 34: The sheath of any example herein, in particular examples 1 to 23, wherein the laced portion, and a plurality of elongate reinforcing portions and at least one elongate foldable portion form respective partial circumferential portions of the expandable shaft portion to extend around a circumference of the expandable shaft portion.
Example 35: The sheath of any example herein, in particular example 34, wherein the laced portion further comprises an elongate foldable portion, and a cord of the laced portion is disposed over an externally oriented surface of an elongate foldable portion.
Example 36: The sheath of any example herein, in particular example 35, wherein the elongate foldable portion of the laced portion comprises elasticity along a lateral dimension.
Example 37: The sheath of any example herein, in particular examples 1 to 36, wherein the sheath is sterilized.
Example 38: A method of delivering a medical device, the method comprising providing an expandable access sheath. The expandable access sheath can comprise a hub and a shaft extending distally from the hub, the shaft comprising an expandable shaft portion. The expandable shaft portion can comprise a laced portion extending along a first partial circumferential portion of the expandable shaft portion, the laced portion comprising a cord configured to form lacing for the laced portion, and a first elongate foldable portion extending along a second partial circumferential portion of the expandable shaft portion. The method can include inserting at least a portion of the expandable shaft portion into a bodily lumen, expanding the laced portion for advancing at least a portion of a medical instrument through a corresponding portion of a shaft delivery lumen extending through the expandable shaft portion, tensioning the cord to collapse the laced portion, and retracting the shaft while the laced portion is in a collapsed configuration.
Example 39: The method of any example herein, in particular example 38, wherein the laced portion comprises lacing coupling adjacent elongate reinforcing portions and wherein expanding the laced portion comprises pushing apart the adjacent elongate reinforcing portions.
Example 40: The method of any example herein, in particular example 39, wherein expanding the laced portion comprises adjusting tension in the cord to allow pushing apart of the adjacent elongate reinforcing portions.
Example 41: The method of any example herein, in particular example 39 or 40, wherein the laced portion comprises a second elongate foldable portion, the cord being disposed over an externally oriented surface of the second elongate foldable portion, and wherein expanding the laced portion comprises unfolding the second elongate foldable portion.
Example 42: The method of any example herein, in particular example 41, wherein expanding the laced portion comprises unfolding and stretching the second elongate foldable portion.
Example 43: The method of any example herein, in particular examples 38 to 42, further comprising unfolding the first elongate foldable portion for advancing the at least a portion of the medical instrument through the corresponding portion of the shaft delivery lumen.
Example 44: The method of any example herein, in particular examples 38 to 42, further comprising unfolding and stretching the first elongate foldable portion for advancing the at least a portion of the medical instrument through the corresponding portion of the shaft delivery lumen.
Example 45: The method of any example herein, in particular examples 38 to 44, wherein tensioning the cord comprises proximally translating proximally disposed portions of the cord.
Example 46: The method of any example herein, in particular example 45, wherein tensioning the cord comprises proximally translating portions of the cord disposed proximally of a proximal end of the hub.
Example 47: The method of any example herein, in particular examples 38 to 46, wherein inserting the expandable shaft portion comprises inserting the expandable shaft portion comprising an introducer preloaded therethrough.
Example 48: The method of any example herein, in particular example 47, wherein inserting the expandable shaft portion comprising the introducer preloaded therethrough comprises maintaining slack in the cord of the laced portion while inserting the expandable shaft portion.
Example 49: The method of any example herein, in particular example 47 or 48, further comprising maintaining the laced portion in the collapsed configuration while inserting the expandable shaft portion comprising the introducer preloaded therethrough.
Example 50: The method of any example herein, in particular example 47, wherein inserting the expandable shaft portion comprising the introducer preloaded therethrough comprises maintaining the laced portion in the collapsed configuration and the cord of the laced portion in a tensioned state while inserting the expandable shaft portion.
Example 51: The method of any example herein, in particular example 47 or 48, wherein inserting the expandable shaft portion comprising the introducer preloaded therethrough comprises maintaining the cord of the laced portion in an expanded configuration while inserting the expandable shaft portion.

The above method(s) can be performed on a living animal or on a simulation, such as on a cadaver, cadaver heart, anthropomorphic ghost, simulator (e.g., with body parts, heart, tissue, etc. being simulated).

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for case of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. An expandable access sheath comprising:
a hub comprising a hub delivery lumen extending therethrough; and
a shaft extending distally from the hub and comprising a shaft delivery lumen extending therethrough and in fluid communication with the hub delivery lumen, the shaft comprising an expandable shaft portion comprising:
a laced portion extending along a first partial circumferential portion of the expandable shaft portion, the laced portion comprising a cord configured to form lacing for the laced portion, and
an elongate foldable portion extending along a second partial circumferential portion of the expandable shaft portion.

2. The sheath of claim 1, wherein the elongate foldable portion comprises elasticity along a lateral dimension.

3. The sheath of claim 1, wherein the laced portion further comprises a second elongate foldable portion along the first partial circumferential portion of the expandable shaft portion, the cord being disposed over an externally oriented surface of the second elongate foldable portion.

4. The sheath of claim 1, further comprising a first and a second elongate reinforcing portion and wherein the cord laces together a first edge of the first elongate reinforcing portion and a second edge of the second elongate reinforcing portion, the first edge and the second edge being configured to be oriented toward one another.

5. The sheath of claim 4, wherein the first and second elongate reinforcing portions each comprises a plurality of openings along a longitudinal dimension, the cord of the laced portion being configured to be passed through the plurality of openings to form the lacing.

6. The sheath of claim 4, wherein a plurality of loops is coupled to each of the first edge of the first elongate reinforcing portion and the second edge of the second elongate reinforcing portion, the cord of the laced portion being configured to be passed through the plurality of loops to form the lacing.

7. The sheath of claim 1, wherein the laced portion is along an entire length of the shaft disposed distally of the hub.

8. The sheath of claim 1, wherein the laced portion is along a proximal portion of shaft.

9. The sheath of claim 1, wherein the expandable shaft portion comprises:
a plurality of elongate reinforcing portions and at least one foldable portion, each of the plurality of elongate reinforcing portions and at least one foldable portion extending around respective portions of a circumference of the expandable shaft portion; and
a plurality of laced portions evenly distributed around and extending around respective remaining portions of the circumference of the expandable shaft portion, each laced portion coupling a respective pair of elongate reinforcing portions.

10. The sheath of claim 9, wherein each of the plurality of laced portions further comprises a corresponding elongate foldable portion, and a cord of each of the laced portions is disposed over an externally oriented surface of the corresponding elongate foldable portion.

11. The sheath of claim 10, wherein each of the corresponding elongate foldable portions of the laced portions comprises elasticity along a lateral dimension.

12. An expandable access sheath comprising:
a hub; and
an expandable shaft extending distally from the hub, the expandable shaft comprising:
a laced portion extending along a first partial circumferential portion, the laced portion comprising a cord configured to form lacing for the laced portion, and
an elongate foldable portion extending along a second partial circumferential portion.

13. The sheath of claim 12, wherein a tension in the cord of the laced portion is configured to be adjustable to change a width of the laced portion.

14. The sheath of claim 12, wherein proximally extending portions of the cord are configured to be disposed proximally of a proximal end of the hub, tension in the proximally extending portions being configured to be adjustable to adjust a tension in the cord.

15. The sheath of claim 12, further comprising a first and a second elongate reinforcing portion and wherein the cord laces together the first and second elongate reinforcing portions.

16. A method of delivering a medical device, the method comprising:
providing an expandable access sheath, the expandable access sheath comprising:
a hub, and
a shaft extending distally from the hub, the shaft comprising an expandable shaft portion, the expandable shaft portion comprising a laced portion extending along a first partial circumferential portion of the expandable shaft portion, the laced portion comprising a cord configured to form lacing for the laced portion, and a first elongate foldable portion extending along a second partial circumferential portion of the expandable shaft portion;
inserting at least a portion of the expandable shaft portion into a bodily lumen;
expanding the laced portion for advancing at least a portion of a medical instrument through a corresponding portion of a shaft delivery lumen extending through the expandable shaft portion;
tensioning the cord to collapse the laced portion; and
retracting the shaft while the laced portion is in a collapsed configuration.

17. The method of claim 16, wherein the laced portion comprises lacing coupling adjacent elongate reinforcing portions, and expanding the laced portion comprises adjusting tension in the cord to allow pushing apart of the adjacent elongate reinforcing portions.

18. The method of claim 16, wherein the laced portion comprises a second elongate foldable portion, the cord being disposed over an externally oriented surface of the second elongate foldable portion, and wherein expanding the laced portion comprises unfolding the second elongate foldable portion.

19. The method of claim 18, wherein expanding the laced portion comprises unfolding and stretching the second elongate foldable portion.

20. The method of claim 16, further comprising one of unfolding the first elongate foldable portion, or unfolding and stretching the first elongate foldable portion, for advancing the at least a portion of the medical instrument through the corresponding portion of the shaft delivery lumen.
